(19) United States
(12) Patent Application Publication
(10) Pub. No.: US 2025/0195212 A1
(43) Pub. Date: Jun. 19, 2025

(54) METHOD FOR GENERATING IN-SITU VASCULAR STENT AND PHOTOCURABLE DRUG-LOADED BALLOON CATHETER FOR IMPLEMENTING THE METHOD

(71) Applicant: HANGZHOU MATRIX MEDICAL TECHNOLOGY CO., LTD., Hangzhou (CN)

(72) Inventor: Peihong Ji, Hangzhou (CN)

(21) Appl. No.: 19/069,124

(22) Filed: Mar. 3, 2025

Related U.S. Application Data
(63) Continuation of application No. PCT/CN2023/110190, filed on Jul. 31, 2023.

(30) Foreign Application Priority Data
Sep. 2, 2022 (CN) 202211073715.0
Nov. 25, 2022 (CN) 202211492006.6
Nov. 28, 2022 (CN) 202211503845.3

(51) Int. Cl. A61F 2/24; A61M 25/10

(52) U.S. Cl. CPC A61F 2/2415; A61M 25/10; A61M 2025/105

(57) ABSTRACT

A method for generating an in-situ vascular stent includes applying a first reagent including riboflavin and/or riboflavin salt to a predetermined location in a blood vessel and applying light to the predetermined location to activate the first reagent, causing the first reagent to act on the predetermined location to generate the in-situ vascular stent.

### CROSS-REFERENCE TO RELATED APPLICATIONS

The present application is a Continuation Application of PCT Application No. PCT/CN2023/110190, filed on Jul. 31, 2023, which claims the priorities of Chinese Patent Application No. 202211073715.0, filed on Sep. 2, 2022, Chinese Patent Application No. 202211492006.6, filed on Nov. 25, 2022, and Chinese Patent Application No. 202211503845.3, filed on Nov. 28, 2022, the entire contents of which are hereby incorporated by reference.

### TECHNICAL FIELD

The present disclosure relates to the field of medical devices, and in particular, to a method for generating an in-situ vascular stent and a photocurable drug-loaded balloon catheter for implementing the method.

### DESCRIPTION OF THE PRIOR ART

In the field of interventional therapy, the balloon dilatation catheter is a commonly used medical device, which is widely used in the dilatation treatment of upper gastrointestinal stricture, cervical dilatation for labor induction, dilation treatment of airway stricture, and dilatation of cardiovascular stenosis areas. In recent years, the drawbacks of traditional balloon dilatation catheters have gradually become apparent, leading to a growing focus on balloons with special properties.

For example, there is a balloon dilatation catheter with a microneedle which is a new type of minimally invasive and nearly painless biomedical device. The microneedle can penetrate the epidermis to form a microchannel, avoiding contact with capillaries and nerve endings. It has the advantages of being minimally invasive, painless, and avoiding infection, while also enabling controlled drug release. The invention patent application with the publication number CN114470341 discloses a composite microneedle balloon and its preparation method, designed to achieve controlled drug release. However, during the catheter dilatation process, the burst pressure of the traditional balloon is relatively low, making it difficult to effectively dilate calcified lesions, especially severely calcified ones. Additionally, at low pressure, the microneedle cannot be effectively pressed into calcified blood vessel walls or the walls of the narrow digestive tract, cervix, airway, etc.

Another example is a balloon dilatation catheter with a photosensitive compound or drug. Different from traditional drug balloons, the balloon body of this type of balloon dilatation catheter can scatter visible light, thereby activating the photosensitive compound. This activation induces rapid binding of collagen and elastin in the blood vessel wall, forming a stent in situ and achieving blood vessel healing and repair. However, in the prior art, the photosensitive compound is relatively dispersed, leading to low efficiency in inducing collagen and elastin binding in the blood vessel wall.

For another example, an angioplasty balloon can form a micro-stent in situ in a blood vessel. The angioplasty balloon is used to open calcified stenotic lesions in the arterial wall. However, after simple balloon dilatation, the likelihood of restenosis at the stenotic lesion site is very high. At present, the angioplasty balloon can be used to open calcified lesions in the arterial wall and is one of the primary methods for arterial stenosis revascularization. However, vascular dilation often causes damage to the blood vessel wall, triggering thrombosis and the release of growth factors, which can lead to restenosis or the subsequent reclosure of the dilated vessel.

At present, the above-mentioned problems are mainly addressed by implanting vascular stents in blood vessels. Existing vascular stents are broadly categorized into two types. The first type is made of biocompatible metals, but it may induce thrombosis and immunogenicity. Additionally, the permanent presence of such stents can interfere with subsequent treatments, potentially causing corrosion, perforation, and the risk of aneurysm formation. The second type consists of biodegradable stents, which address the issue of the permanent presence of metal stents. However, the acidic degradation products of these stents can cause severe inflammatory reactions and lead to the atrophy and degradation of the muscle elastic elements of the arterial wall, resulting in arterial dilation. To reduce the restenosis rate of diseased vessels after stent implantation, drug-eluting stents are widely used. While drug-eluting stents can reduce the proliferation of vascular smooth muscle cells and vascular restenosis, they also prevent the long-term restoration of the endothelial cell layer, thereby causing thrombosis in the blood vessel wall.

### SUMMARY OF THE DISCLOSURE

In view of the above, the present invention provides a drug-loaded balloon catheter and preparation method thereof, a balloon catheter system and method for generating in-situ vascular stent, to improve the drug delivery effect of the drug-loaded balloon catheter.

A drug-loaded balloon catheter, including:
a tube body having opposite distal and proximal ends for delivering fluid into a balloon body; and
a balloon body, fixed to the distal end of the tube body and in communication with the tube body, the balloon body being a hollow structure and having an inflated state and a deflated state suitable for interventional delivery;
wherein the balloon body is loaded with a drug in one of the following methods: solid embedding, solid coating, and solution infiltration.

Several optional methods are further provided below, but they are not intended to be additional limitations on the above-mentioned overall solution, but are merely further supplements or preferences. Without technical or logical contradictions, each optional method can be combined with the above-mentioned overall solution separately, and multiple optional methods can also be combined.

Optionally, the balloon body is loaded with a drug in a solid embedding manner, and the drug-loaded balloon catheter further includes:
a braided mesh, which is made of polymer material and wraps around the balloon body, the braided mesh having a plurality of cells, the cell having a width of X and a length of Y, wherein X:Y=1:0.5 to 2; the area S of the cell is in the range of 1 to 50 mm²; and
drug-loaded microneedles, which are arranged on the surface of the balloon body and embedded with drugs, and are located inside the cells of the braided mesh and/or at the interweaving points of the cells.

Optionally, a plurality of microneedles are distributed in each cell, the distance between any two adjacent microneedles in the cell is in the range of 30 μm to 3 mm, and the height of the microneedles is in the range of 25 to 2,000 μm.

Optionally, the braided mesh comprises:
a first weaving filament, which is helically wound around the outer circumference of the balloon body; and
a second weaving filament, which extends axially along the balloon body and interweaves with the first weaving filament, wrapping around the corresponding loops of the first weaving filament at least once at the corresponding interweaving points.

Optionally, the filament of the braided mesh is helically wound around the outer circumference of the balloon body, and two adjacent loops are respectively a first winding loop and a second winding loop, and each winding loop has alternating crests and troughs that are interconnected.

Optionally, the braided mesh comprises:
a plurality of first filaments arranged in parallel, each of which extends circumferentially along the balloon body, and the first filaments are arranged at intervals in the axial direction of the balloon body; and
a second filament which extends along the axial direction of the balloon body and interweaves with the first filaments, wrapping around the corresponding first filaments at least once at the corresponding interweaving points.

Optionally, the balloon body is loaded with a drug in the form of a solid coating, and the drug-loaded balloon catheter further includes:
an optical fiber assembly which is inserted into the tube body and has a light-emitting portion extending to the position adjacent to the balloon body.

The surface of the balloon body is loaded with an adjuvant and a photosensitizer in the form of a coating, and the photosensitizer is a polypeptide dendrimer modified with a naphthalimide compound.

The photosensitizer can activate collagen and elastin at a light wavelength in the range of 400 to 460 nm, inducing cross-linking.

The adjuvant includes an active drug and a sustained-release material encapsulating the active drug, and the active drug is at least one of paclitaxel, rapamycin, zotarolimus, tacrolimus, everolimus, temsirolimus, biolimus, docetaxel, protein-bound paclitaxel and protein-bound dexamethasone.

Optionally, the preparation method of the photosensitizer includes:
step 1, protecting part of the amino groups of the polypeptide dendrimer; and
step 2, adding the polypeptide dendrimer with part of the amino groups protected and a naphthalimide compound to a mixed solution of an organic base and an organic solvent, reacting at 70 to 150° C. for 1 to 32 hours, followed by post-processing to obtain the photosensitive material.

The organic base is at least one of N, N-diisopropylethylamine, sodium tert-butoxide, and potassium tert-butoxide.

The organic solvent is at least one of isopropanol, hexafluoroisopropanol, methanol, tetrahydrofuran, dioxane, acetonitrile, ethyl acetate, dichloromethane, dimethyl sulfoxide, N, N-dimethylacetamide, N-methylpyrrolidone and hexamethylphosphoramide.

Optionally, the surface of the balloon body is loaded with a photosensitizer in the form of a coating, including the following steps: dispersing or dissolving the photosensitizer in a solvent to prepare a solution, and coating the solution on the surface of the balloon body.

The solvent is at least one of ethanol, acetic acid, acetone, butylated hydroxytoluene, methyl ethyl ketone, ethyl acetate, tetrahydrofuran and water.

Optionally, the solvent is a mixture of ethanol, acetic acid and water, and the volume ratio of these components in the solvent is ethanol:acetic acid:water=80 to 90:19 to 9:1.

Optionally, in the solution, the concentration of the photosensitizer is in the range of 6.25 to 125 μM/mL.

Optionally, the solution further includes an adjuvant, and the mass ratio of the adjuvant to the photosensitizer is in the range of 0.3 to 10.

Optionally, for the adjuvant, the mass ratio of the active drug to the sustained-release material is in the range of 1:1 to 20.

Optionally, the mass ratio of the photosensitizer to the active drug is in the range of 1:0.2 to 5.

Optionally, the solution further includes a stabilizer, wherein the stabilizer is at least one of an antioxidant and a Lewis acid.

The antioxidant is at least one of tromethamine and butylated hydroxytoluene. The mass ratio of the antioxidant to the photosensitizer is in the range of 0.05 to 1:100;

The cation of the Lewis acid is at least one of Na⁺, K⁺, Mg²⁺, and Ca²⁺. The molar ratio of the Lewis acid to the photosensitizer is in the range of 0.8 to 3.

Optionally, the coating method includes spraying and/or dipping, and the photosensitizer coating amount on the balloon surface is in the range of 0.0012 to 37.5 μM/mm².

Optionally, the outer surface of the balloon body is loaded with a drug coating, and the active ingredient in the drug coating includes riboflavin and/or riboflavin salt.

The drug-loaded balloon catheter further includes: a light-guiding element, one end of which is a light-emitting end extending to the balloon body, and the other end of which is a light-incident end extending through the tube body to the proximal end.

Optionally, the drug coating is loaded by:
preparing a solution for drug coating in advance, coating the solution on the surface of the balloon body, and then drying it.

Optionally, the balloon body is loaded with the drug by solution infiltration, and the drug-loaded balloon catheter further includes:
the balloon wall of the balloon body has a pore structure;
a fluid for maintaining the balloon body in the inflated state, outputting to a surrounding environment of the balloon body through the pore structure, wherein the fluid contains riboflavin and/or riboflavin salt;
a light-guiding element having one end as a light-emitting end extending to the balloon body and the other end as a light-incident end extending to the proximal end through the tube body.

Optionally, the pore size of the pore structure is in a range of 5 to 100 μm.

The surface porosity of the balloon body is in a range of 30% to 80%.

Optionally, the fluid is in the form of a solution, and its solvent is water.

The concentration of the fluid is in a range of 0.2 to 60 mg/mL calculated based on total riboflavin.

The present disclosure further provides a method for preparing the drug-loaded balloon catheter, including the following steps:
filling the micropores of the rigid substrate with the drug-containing raw material liquid of the microneedles and in-situ forming to obtain the microneedles;
wrapping the braided mesh around the balloon body;
injecting fluid into the balloon body to expand the balloon body; and
coating an adhesive on the balloon body or the microneedles, rolling the balloon body on the rigid substrate, and adhering the microneedles to the surface of the balloon body.

Optionally, the solvent of the microneedle raw material liquid is water, and the solute is at least one of chitosan, sodium alginate, polyethylene glycol, PLGA, PCL, PMMA, PGA, PLA, PEA, gelatin, and hyaluronic acid.

Optionally, the adhesive is a re-dissolvable adhesive that dissolves in water.

Optionally, before or after fluid is injected into the balloon body to expand the balloon body, a core shaft is inserted into the balloon body, and the core shaft is operated to drive the balloon body to roll on the rigid substrate.

The present disclosure further provides a method for preparing the drug-loaded balloon catheter, including the following steps:
wrapping the braided mesh around the balloon body;
injecting fluid into the balloon body to expand the balloon body;
dripping the ultraviolet-curable adhesive onto the surface of the balloon body or the interweaving points of the cells of the braided mesh, and then applying an electric field or a magnetic field in a specific direction to make the ultraviolet-curable adhesive take on the shape of microneedles;
irradiating with ultraviolet light to cure the ultraviolet-curable adhesive in the shape of microneedles to form microneedles; and
spraying or dip-coating drug on the surface of the microneedles.

The present disclosure further provides a balloon catheter system, including:
a balloon body having an inflated state and a deflated state suitable for interventional delivery, the balloon wall of the balloon body having a pore structure for fluid to pass through;
a tube body having a distal end and a proximal end opposite to each other, wherein the distal end is connected to the balloon body;
a drug delivery device connected to the proximal end of the tube body and configured for supplying fluid, wherein the fluid contains riboflavin and/or riboflavin salt;
a light-guiding element, one end of which is a light-emitting end extending to the balloon body, and the other end of which is a light-incident end extending to the proximal end through the tube body; and
a light source device which is connected to the light-incident end of the light-guiding element by an optical path.

The present disclosure further provides a method for generating an in-situ vascular stent, including:
applying a first reagent including riboflavin and/or riboflavin salt to a predetermined location in a blood vessel; and
applying light to the predetermined location to activate the first reagent, causing the first reagent to act on the predetermined location to generate the in-situ vascular stent.

Optionally, applying the first reagent includes:
preparing a solution and delivering it to the predetermined location through an interventional device; or
using coating or solid embedding and delivering it to the predetermined location through the interventional device.

When the first reagent is in the form of a solution, the concentration of the first reagent is in a range of 0.2 to 60 mg/mL calculated based on total riboflavin.

Optionally, the wavelength of the applied light is in a range of 300 to 700 nm; the light intensity is in a range of 5 to 500 mW/cm²; and the duration of applying light is in a range of 0.1 to 30 minutes.

The present disclosure further provides use of riboflavin and riboflavin salt in the preparation of an in-situ vascular stent drug, including applying the riboflavin and/or riboflavin salt to a predetermined location and then generating an in-situ vascular stent at the predetermined location by photoexcitation.

The drug-loaded microneedle balloon dilatation catheter provided in the present disclosure can effectively dilate calcified blood vessels, especially severely calcified blood vessels. When the balloon body is pressurized, the microneedles can be effectively pressed into the calcified blood vessel walls or the walls of the narrow digestive tract, cervix, airway, etc., and the drug can be slowly released for treatment.

The balloon catheter system provided in the above embodiments improves the photosensitizer structure and coverage on the surface of the balloon body, thereby increasing the utilization efficiency of the photosensitizer compound.

In the present disclosure, riboflavin is transported or delivered to the blood vessel wall via an interventional device. Then, riboflavin is activated by light, enabling it to cross-link with proteins and polypeptides in the blood vessel wall. As a result, an endogenous micro-stent is generated in situ on the blood vessel wall to replace the implantable stent. This can effectively reduce the formation of thrombus and immunogenicity. The micro-stent formed in situ can maintain the blood vessel in the dilated shape after angioplasty and prevent restenosis.

### BRIEF DESCRIPTION OF DRAWINGS

FIG. 1 is a schematic view of a drug-loaded balloon catheter according to one embodiment of the present disclosure;

FIG. 2 is an enlarged view of a balloon body of the drug-loaded balloon catheter according to one embodiment of the present disclosure;

FIG. 3 is a schematic view of a microneedle of the drug-loaded balloon catheter according to one embodiment of the present disclosure;

FIG. 4a is a schematic view of a first braided mesh of the drug-loaded balloon catheter of the present disclosure;

FIG. 4b is a schematic view of a first braided mesh of the drug-loaded balloon catheter of the present disclosure with the microneedles not shown;

FIG. 5 is a schematic view of a second braided mesh of the drug-loaded balloon catheter of the present disclosure;

FIG. 6 is a schematic view of a third braided mesh of the drug-loaded balloon catheter of the present disclosure;

FIG. 7 is a schematic view of a substrate with micropores used in the preparation process of the first preparation method of the drug-loaded balloon catheter according to one embodiment of the present disclosure;

FIG. 8 is a schematic view showing the state after threading the core shaft into the balloon body and rolling to bond the microneedles during the preparation process of the first preparation method of the drug-loaded balloon catheter according to an embodiment of the present disclosure;

FIG. 9 is a schematic view showing the state after threading the core shaft into the balloon body and rolling to bond the microneedles during the preparation process of the first preparation method of the drug-loaded balloon catheter according to an embodiment of the present disclosure (the braided mesh is different from that shown in FIG. 8);

FIG. 10 is a schematic view showing a second preparation method of the drug-loaded balloon catheter of the present disclosure (the microneedles are located at the interweaving points of cells of the braided mesh);

FIG. 11 is a schematic view of a drug-loaded balloon catheter system according to an embodiment;

FIG. 12 is a tissue staining image of the experimental group in the characterization of the blood vessel repair effect;

FIG. 13 is a tissue staining image of the control group in the characterization of the blood vessel repair effect;

FIG. 14 is a flow chart of a method for generating an in-situ vascular stent according to the present disclosure;

FIG. 15 is a schematic view of a drug-loaded balloon catheter based on photocuring according to an embodiment;

FIG. 16 is a schematic view of a drug-loaded balloon catheter for an in-situ vascular stent according to an embodiment;

FIG. 17 is a schematic view of a photocurable balloon catheter system for generating an in-situ vascular stent according to an embodiment;

FIG. 18 is a schematic view of a blocking balloon catheter according to an embodiment.

### LIST OF REFERENCE NUMBERS

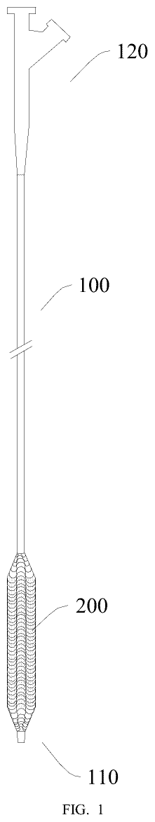

in FIGS. 1 to 10:
100, tube body; 110, distal end; 120, proximal end; 200, balloon body; 210, constant-diameter section; 220, reduced-diameter section; 300, braided mesh; 311, first weaving filament; 312, second weaving filament; 321, first cell; 322, second cell; 400, microneedle; 500, substrate; 510, micropore; 600, core shaft; 700, electric field.
X, cell width; Y, cell length; Y1, first cell length; Y2, second cell length; S, cell area; C1, first winding loop; A1, crest; B1, trough; C2, second winding loop; A2, crest; B2, trough; W1, crest width; W2, trough width; H1, crest spacing; L1, first filament; L2, second filament; Z, interweaving point;

in FIG. 11:
100, tube body; 200, balloon body; 130, guidewire lumen; 140, perfusion lumen; 160, inner tube; 170, outer tube; 340, optical fiber assembly; 310, light-emitting device; 320, optical fiber body; 330, light-emitting portion;

in FIGS. 14 to 18:
10, balloon body; 11, balloon wall; 12, pore structure; 13, first balloon body; 14, second balloon body; 20, tube body; 21, proximal end; 22, distal end; 30, light-guiding element; 31, light-emitting end; 32, light-incident end; 40, drug delivery device; 50, light source device; 60, drug coating; 70, blood vessel; 71, blood vessel wall.

### DESCRIPTION OF EMBODIMENTS

The technical solutions according to the embodiments of the present disclosure will be described clearly and fully in combination with the drawings according to the embodiments of the present disclosure. Obviously, the described embodiments are not all embodiments of the present disclosure, but only part of the embodiments of the present disclosure. Based on the disclosed embodiments, all other embodiments obtained by those skilled in the art without creative work fall into the scope of this disclosure.

In order to better describe and illustrate the embodiments of the present disclosure, one or more accompanying drawings may be referenced. However, the additional details or examples provided in the description of the drawings should not be construed as limiting the scope of the disclosure, the embodiments currently described, or any of the preferred implementations disclosed herein.

It should be noted that, when a component is “connected” with another component, it may be directly connected to another component or may be indirectly connected to another component through a further component. When a component is “provided” on another component, it may be directly provided on another component or may be provided on another component through a further component.

Unless otherwise defined, all technical and scientific terms used herein have the same meaning as commonly understood by those skilled in the art to which this disclosure pertains. The terminology used in the specification of this disclosure is solely for the purpose of describing specific embodiments and is not intended to limit the scope of the disclosure.

The present disclosure provides a drug-loaded balloon catheter, including:
a tube body having opposite distal and proximal ends for delivering fluid into a balloon body; and
the balloon body which is fixed to the distal end of the tube body and in communication with the tube body, the balloon body being a hollow structure and having an inflated state and a deflated state suitable for interventional delivery;
wherein the balloon body is loaded with a drug in one of the following methods: solid embedding, solid coating, and solution infiltration.

In the present disclosure, the balloon body of the balloon catheter is loaded with a drug in various methods. In addition to the different loading methods, the types of drugs corresponding to different loading methods are also different.

First, referring to FIGS. 1 to 10, a detailed description is provided for the embodiment in which the drug is loaded in the form of solid embedding. In simple terms, the solid embedding method involves fixing drug-containing microneedles on the surface of the balloon body. The drug-loaded balloon catheter obtained through this loading method is referred to as a drug-loaded microneedle balloon dilatation catheter, and its preparation method is also described in detail.

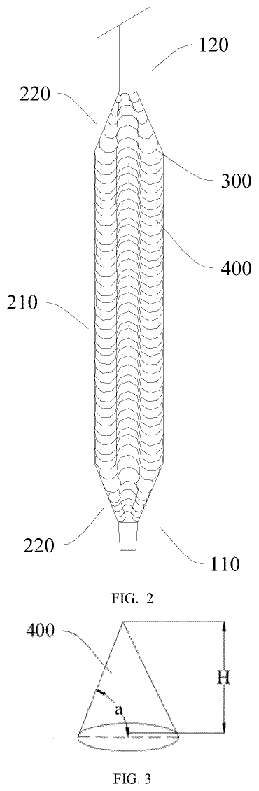

Referring to FIG. 1 and FIG. 2, a drug-loaded microneedle balloon dilatation catheter includes:
a tubular body 100 having a distal end 110 and a proximal end 120 opposite to each other;
a balloon body 200 fixed to the distal end 110 of the tube body 100 and having a hollow structure;
a braided mesh 300 made of polymer material and wrapping around the outer periphery of the balloon body 200, the braided mesh having a plurality of cells, the width of the cell being X, the length being Y, wherein X:Y=1:0.5 to 2; the area S of the cell being in the range of 1 to 50 mm²; and
drug-loaded microneedles 400 disposed on the surface of the balloon body 200 and located inside the cells and/or at the interweaving points of the cells of the braided mesh 300.

In the present disclosure, the outside of the balloon body 200 is wrapped with a braided mesh 300, and the braided mesh 300 has cells. When the balloon body 200 expands under high pressure, the braided mesh 300 can constrain the deformation of the balloon body 200, ensuring uniform expansion of the balloon body 200 and reducing the risk of blood vessel tearing. Meanwhile, when the balloon body 200 expands under high pressure, under the constraint of the braided mesh, the outer peripheral areas of the balloon body 200 within the cells bulge outward, and the microneedles 400 are pressed into the diseased vessel wall, allowing for the slow release of the drug in the diseased vessel wall.

Referring to FIG. 2, the balloon body 200 includes a constant-diameter section 210 in the middle and tapered reduced-diameter sections 220 at both ends in the axial direction. The ends of the two reduced-diameter sections 220 away from the constant-diameter section 210 converge to the tube body 100. The material of the balloon body 200 can be at least one of PA, Pebax, and PU.

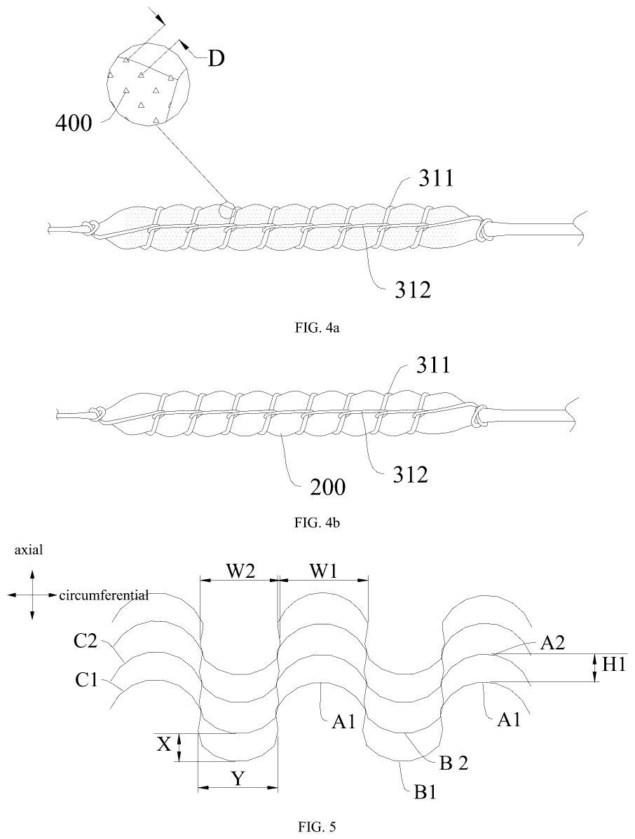

As shown in FIGS. 1, 2, 3 and 4, a plurality of microneedles 400 are distributed in the cells, and the distance (D in FIG. 4a) between any two adjacent microneedles 400 within the cell is in the range of 30 μm to 3 mm, and the height (H in FIG. 3) of the microneedle 400 is in the range of 25 to 2000 μm. Preferably, the distance between any two adjacent microneedles 400 is in the range of 1 mm to 3 mm, and the height of the microneedle 400 is in the range of 25 to 10 00 μm. More preferably, the distance between any two adjacent microneedles 400 is in the range of 1 mm to 3 mm, and the height of the microneedle 400 is in the range of 100 to 500 μm.

Microneedles 400 can be distributed inside the cells of the braided mesh, or at the interweaving points of the cells, or both inside the cells and at the interweaving points of the cells. There is no significant difference between these distribution positions.

When setting the spacing and height of the microneedles 400, the limitations of the processing technology need to be considered while maximizing the drug-loading capacity as much as possible. Additionally, the setting must consider the bearing capacity of the blood vessel after the microneedles penetrate the blood vessel.

As shown in FIG. 3, the microneedle 400 has a sharp tip capable of penetrating the target object. However, it should not be excessively sharp to prevent damage to the balloon wall of the balloon body 200 when folded and inserted into the human body. As an embodiment, the microneedle 400 is conical. The diameter of the base of the cone ranges from 30 to 1000 μm, and the degree of the base angle a of the cone is 45°≤a<90°. Further, the degree of the base angle a of the cone is 45°≤a<70°. The microneedle 400 can also adopt other shapes, such as a multi-faceted pyramid. Alternatively, various existing microneedle designs can be used, including solid microneedles 400 and multilayer microneedles 400.

The braided mesh 300 is woven from filamentous materials. The cross-section of a single filamentous material can be circular with a diameter in the range of 10 to 200 μm. Alternatively, the cross-section of the filamentous material can be rectangular with a length in the range of 10 to 300 μm and a width in the range of 10 to 100 μm.

The filamentous material is made of high-strength medical-grade polymer material to meet the high-pressure filling requirements in the balloon body 200, ensuring safety and effectiveness during pressurization of the balloon body 200. Examples include nylon, polyether-block-amide, polytetrafluoroethylene, polyethylene (ultra-high molecular weight polyethylene), etc. The filamentous materials can be directly woven or twisted before being woven.

The braided mesh 300 can be fixed to the outer surface of the balloon body 200 by bonding. Adhesives such as polyurethane and polyvinyl chloride can be used for the bonding. The curing method of the adhesive can be natural curing or UV curing. The materials and bonding methods used for the braided mesh 300 in this disclosure can effectively improve the flexibility of the balloon body 200.

The first weaving method of the braided mesh is shown in FIG. 4a and FIG. 4b. The braided mesh includes:
a first weaving filament 311, which is helically wound around the outer circumference of the balloon body 200; and
a second weaving filament 312, which extends axially along the balloon body 200 and interweaves with the first weaving filament 311, wrapping around the corresponding loops of the first weaving filament 311 at least once at the corresponding interweaving points.

The first weaving filament 311 is helically wound around the balloon body 200 from one end to the other end. During the expansion of the balloon body 200, force transmission is more direct, ensuring that the braided mesh 300 maintains good overall integrity and coordination and allows for a higher expansion pressure. That is, even at high pressures, the expansion is uniform.

The second weaving filament 312 is used to limit the first weaving filament 311 and prevent the loops of the first weaving filament 311 from stacking in the axial direction due to uneven force.

Referring to FIG. 4a, after the balloon body 200 is filled with liquid, under the constraint of the braided mesh, the outer peripheral wall of the balloon body within the cells bulges slightly outward, pressing the microneedles thereon into the blood vessel or plaque.

As shown in FIG. 2, a single filament is wound in the axial direction, W1 is the crest and W2 is the trough. Alternatively, a straight line course can be adopted. L1 and L2 are adjacent widths, L1:L2=1.2:1 to 1:1.2, the number of L1 is N, where N is in the range of 2 to 6, the number of L2 is in the range of N−1 to N+1. H1 is in the range of 1.5 mm to 5 mm.

The second weaving method of the braided mesh is shown in FIG. 5. The width of each cell of the braided mesh 300 is X, the length is Y, and X:Y=1:0.5 to 2. The area of each cell is in the range of 1 to 50 mm². The cell with a larger area ensures that the balloon body 200 can form distinct bulges during expansion. The width, length and area of each cell are measured on the flattened plane of the braided mesh 300.

Furthermore, in each cell, X:Y=1:1 to 1.5, and the cell of this size ratio is roughly “short and wide”, which can constrain the balloon body 200 to form a distinct bulge, and the contact area between the bulge and the blood vessel is increased.

The area of each cell is preferably in the range of 2 to 38 mm², so that when the balloon body 200 expands, the balloon body 200 can be constrained to form larger bulges.

There are many weaving methods for the braided mesh 300. For example, in one embodiment, the filament of the braided mesh 300 is helically wound around the outer periphery of the balloon body 200. The two adjacent loops are the first winding loop C1 and the second winding loop C2. Each winding loop has alternating crests and troughs that are interconnected. As shown in FIG. 5, the first winding loop C1 has connected crests A1 and troughs B1, and the second winding loop C2 has connected crests A2 and troughs B2. In this embodiment, the crests and troughs are positioned oppositely, and they can constrain the balloon body 200 to form distinct bulges and grooves during the expansion of the balloon body 200.

Furthermore, in two adjacent loops, the trough B2 of the second winding loop C2 spans across the two adjacent crests A1 of the first winding loop C1. The positions of the troughs are aligned, and the positions of the crests are aligned. Compared with the case where in two adjacent winding loops, the crests of one loop are intertwined with the two adjacent troughs of the other loop, the cell size of the braided mesh 300 in this embodiment is more uniform, and during the expansion of the balloon body 200, the balloon body 200 can be constrained to form relatively uniform bulges and grooves.

In order to form effective bulges and grooves, in one embodiment, the width of the crest is W1, the width of the trough is W2, and W1:W2 is 1:0.8 to 1.2. In addition, the crest spacing H1 between two adjacent loops is in the range of 1.5 to 5 mm. The crests and troughs appear in pairs, which can be 1 to 6 pairs. Referring to FIG. 5, in this embodiment, each winding loop is arranged along the circumference of the balloon body 200, with an inflection point between the crest and the trough. The width of a single crest (or trough) is the straight-line distance between the inflection points at both ends. The length Y of each cell is approximately equal to the width W1 of the crest, and the width X is approximately equal to the crest (or trough) spacing H1.

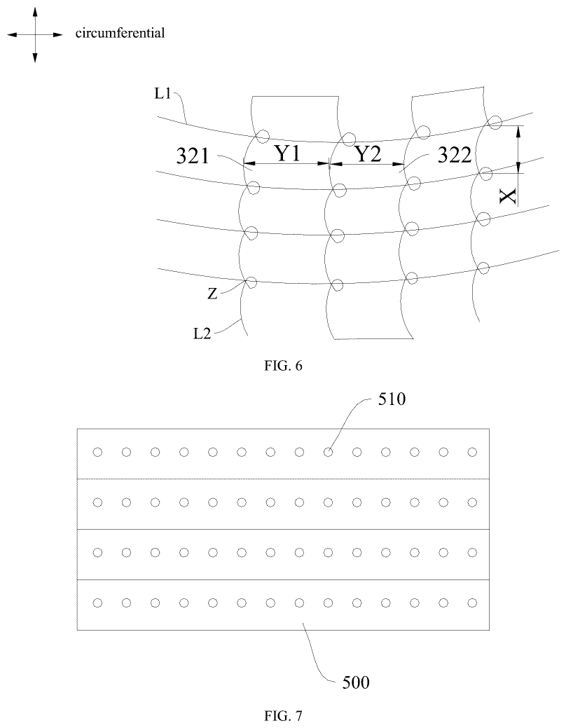

The third weaving method for the braided mesh is shown in FIG. 6. Another embodiment further provides a weaving method, wherein the braided mesh 300 includes a plurality of first filaments L1 and a second filament L2. The first filaments L1 are arranged in parallel, and each first filament L1 extends circumferentially along the balloon body 200. The first filaments L1 are arranged at intervals in the axial direction of the balloon body 200, and the axial interval distance is the width X of the cell. To form a plurality of cells, the second filament L2 extends axially along the balloon body 200 and interweaves with the first filaments L1, wrapping around the corresponding first filaments L1 at least once at the corresponding interweaving points Z. The spacing between the two interweaving points Z of a single first filament L1 is the length Y of the cell. The axial and circumferential directions of the balloon body 200 referred to in this embodiment are relative concepts and are interchangeable. The cell formed by the weaving method in this embodiment is roughly rhombus or rectangular, and its four corners are the interweaving points Z of the first filaments L1 and the second filament L2. During the expansion of the balloon body 200, the cells constrain the balloon body 200 to form bulges, and these interweaving points Z create gaps between these bulges.

Furthermore, in the axial direction of the balloon body 200, the lengths of two adjacent cells (the first cell 321 and the second cell 322) of the braided mesh 300 are Y1 and Y2, respectively, and Y1:Y2 is in the range of 1:0.8 to 1.2.

For a single balloon body 200, if the cells of the braided mesh 300 are too dense, the cells will be relatively small (compared to a sparser braided mesh 300), making it difficult to form bulges and resulting in an insignificant effect of the drug-loaded microneedle balloon dilatation catheter. Conversely, if the cells of the braided mesh 300 are too sparse, fewer bulges will be formed, making it harder to effectively distribute the pressure of the balloon body 200 on the vascular plaque. Therefore, an appropriate number of cells needs to be set. For example, in one embodiment, a balloon body 200 with a diameter of 4 mm and a length of 5 cm is used.

Generally, the number of cells of the braided mesh 300 in the axial direction of the balloon body 200 is required to be in the range of 10 to 34, and the number of cells of the braided mesh 300 in the circumferential direction of the balloon body 200 is in the range of 2 to 16.

The drug-loaded microneedle balloon dilatation catheter provided in the present disclosure uses a high-strength braided mesh 300 to constrain the balloon body 200. The cell area of the braided mesh 300 is large, and distinct bulges can be formed under the high pressure of the balloon body 200, with gaps between bulges, effectively shaping the balloon body 200 and enabling it to expand severely calcified blood vessels.

The present disclosure further provides a method for preparing a drug-loaded microneedle balloon dilatation catheter, including the following steps:
filling the micropores 510 of the rigid substrate 500 with the drug-containing raw material liquid of the microneedles 400 and in-situ forming to obtain the microneedles 400;
wrapping the braided mesh around the balloon body 200;
injecting fluid (which may be liquid or gas) into the balloon body 200 to expand the balloon body 200; and
coating an adhesive on the balloon body 200 or the microneedles 400, rolling the balloon body 200 on the rigid substrate 500, and adhering the microneedles 400 to the surface of the balloon body 200.

The structure of the rigid substrate 500 is shown in FIG. 7. The rigid substrate 500 is made of silicon material, and the thickness of the substrate 500 is in the range of 1200 to 1500 μm. The shape and height of the micropore 510 on the substrate 500 are the same as those of the microneedle 400, that is, the micropore 510 serves as the mold cavity for the microneedle 400. After the raw material liquid of the microneedle 400 is injected into the micropore 510, it is cured in situ to form the microneedle 400.

When filling the micropores 510 of the rigid substrate 500 with the drug-containing raw material liquid of the microneedles 400, the rigid substrate 500 with the micropores 510 can be immersed in the drug-containing raw material liquid of the microneedles 400, and the micropores 510 can be filled with the drug-containing raw material liquid of the microneedles 400 by centrifugation or pressurization. The rigid substrate 500 is dried (through vacuum drying, oven drying, freeze drying, etc.) to form the microneedles 400 in situ in the micropores 510. If freeze drying or vacuum drying is used, a porous structure will be formed inside the microneedle 400, which is conducive to the release of the drug in the microneedle 400.

The micropores 510 on the rigid substrate 500 not only have the same shape and height as the microneedles 400, but the arrangement of the micropores 510 is also the same as the expected arrangement of the microneedles 400 on the balloon body 200. That is, when rolling the balloon body 200 on the rigid substrate 500, the positions where the microneedles 400 are bonded are exactly within the target cells.

The steps of wrapping the braided mesh 300 around the balloon body 200 and injecting fluid into the balloon body 200 to expand the balloon body 200 can be interchanged in order, and the braided mesh 300 and the balloon body 200 can be fixed by bonding.

The solvent of the raw material liquid of the microneedle 400 is water, and the solute is at least one of chitosan, sodium alginate, polyethylene glycol, PLGA, PCL, PMMA, PGA, PLA, PEA, gelatin, and hyaluronic acid. The drug content in the raw material liquid of the microneedle 400 is in the range of 1 to 10 wt %. More preferably, the drug content in the raw material liquid of the microneedle 400 is in the range of 1 to 5 wt %.

The drug-loaded microneedles 400 are bonded to the outer surface of the balloon body 200 with a water-soluble adhesive. Upon contact with blood inside the human body, the adhesive rapidly dissolves, allowing the microneedles 400 to detach from the outer surface of the balloon 200 and remain embedded in the blood vessel wall. For example, the water-soluble adhesive can be a polyethylene glycol hydrogel.

In the present disclosure, the burst pressure of the balloon body 200 is in the range of 30 to 45 atm, preferably in the range of 30 to 40 atm. When the burst pressure exceeds 40 atm, all narrow blood vessels can be expanded. After the balloon body expands the blood vessel, the microneedles 400 penetrate the vessel wall, and the balloon body 200 is slightly rotated to ensure that the drug-loaded microneedles 400 remain embedded in the vessel wall for sustained drug release.

In practice, before the balloon body 200 expands, the microneedles 400 are hidden in the folds of the balloon body 200. After the balloon body 200 expands, the microneedles 400 penetrate the vessel wall for 0.2 to 10 minutes. Then, the balloon body 200 is depressurized to the nominal pressure of −0.5 atm, and blood enters the gap between the balloon body 200 and the vessel wall to dissolve the adhesive fixing the microneedles 400, causing the microneedles 400 to fall off from the wall of the balloon body 200 and then remain on the vessel wall to achieve sustained drug release.

The microneedles 400 fall off from the balloon body 200 and remain in the lesion site, slowly releasing the drug for treatment. The microneedles can degrade over a predetermined period to avoid adverse effects on the human body.

The raw material liquid of microneedle 400 is preferably made of a material that is degradable in the human body. The microneedles 400 remain in the blood vessels or plaques to slowly release the drug. After the drug is released, the microneedles 400 can be degraded. The degradation time varies depending on the material.

In one embodiment, for example, chitosan is first dissolved in a 1% (by mass) acetic acid solution (or pH=5.5±0.5) to obtain a 3% (by mass) chitosan solution. An acid-soluble drug is dissolved in the chitosan solution. A rigid substrate 500 with micropores 510 is placed in the chitosan solution, the rigid substrate 500 is kept completely immersed in the solution, and centrifuged at 8000 rpm for 5 min. After that, the rigid substrate 500 is taken out, with the excess chitosan solution on the surface of the rigid substrate 500 scraped off, and freeze-dried at −80° C. to obtain drug-loaded microneedles 400.

The adhesive is a re-dissolvable, water-soluble medical adhesive, such as polyethylene glycol hydrogel. After encountering blood in the human body, within 10 minutes, the polyethylene glycol hydrogel dissolves, and the microneedles 400 penetrate the inner wall of the blood vessel and slowly release the drug in the inner wall of the blood vessel.

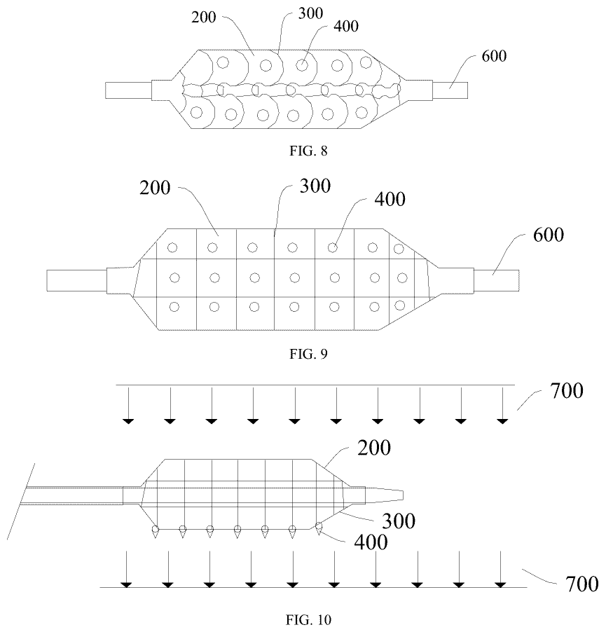

Referring to FIGS. 8 and 9, before or after fluid is injected into the balloon body 200 to expand the balloon body 200, a core shaft 600 is inserted into the balloon body 200, and the core shaft 600 is operated to drive the balloon body 200 to roll on the rigid substrate 500.

The core shaft 600 is operated to drive the balloon body 200 to roll on the rigid substrate 500, and the microneedles 400 are bonded to the surface of the balloon body 200. FIGS. 8 and 9 specifically illustrate the arrangement of microneedles 400 within the individual cells, with a sparse distribution for clarity. In practice, the actual number of microneedles 400 is significantly higher, and the density of their arrangement can be referred to FIG. 4a.

The present disclosure further provides a method for preparing a drug-loaded microneedle balloon dilatation catheter, including the following steps:
wrapping the braided mesh 300 around the balloon body 200;
injecting fluid (which may be liquid or gas) into the balloon body 200 to expand the balloon body 200;
dripping the ultraviolet-curable adhesive onto the surface of the balloon body 200 or the interweaving points of the cells of the braided mesh 300, and then applying an electric field 500 or a magnetic field in a specific direction to make the ultraviolet-curable adhesive take on the shape of microneedles;
irradiating with ultraviolet light to cure the ultraviolet-curable adhesive in the shape of microneedles to form microneedles; and
spraying or dip-coating drug on the surface of the microneedles.

Since the balloon body 200 generally has a cylindrical structure, the direction of the electric field or magnetic field remains constant, and the ultraviolet-curable adhesive on the balloon body 200 can be formed into microneedle shapes by rotating the balloon body 200. After the microneedle shape is formed, the ultraviolet-curable adhesive is cured and shaped, and then the balloon body is rotated to the next position. Alternatively, the ultraviolet-curable adhesive on the balloon body 200 can be formed into microneedle shapes by changing the direction of the electric field or magnetic field. After the microneedle shape is formed, the ultraviolet-curable adhesive is cured, and then the direction of the electric field or magnetic field is changed.

Since the ultraviolet-curable adhesive needs to form microneedle shapes under the action of an electric field, the ultraviolet-curable adhesive needs to be conductive, that is, it needs to be sensitive to electric field signals. For a non-conductive ultraviolet-curable adhesive, 5 to 30% of conductive materials (such as graphite, iron powder, and aluminum powder) can be added to make it conductive.

The ultraviolet-curable adhesive may be at least one of epoxy acrylate adhesive, polyurethane acrylate adhesive, polyether acrylate adhesive, polyester acrylate adhesive, and acrylic resin adhesive.

The volume of each drop of ultraviolet-curable adhesive is in the range of 0.5 to 3 μL, and the ultraviolet-curable adhesive can be dropped within the cells of the braided mesh, or it can be dropped at the interweaving of the cells of the braided mesh.

The ultraviolet-curable adhesive is formed into microneedle shapes under the action of an electric field or a magnetic field. The intensity of the electric field or the magnetic field is determined according to the flowability and conductivity of the ultraviolet-curable adhesive. At a specific flowability, applying an appropriate field intensity can stretch the ultraviolet-curable adhesive into microneedles ranging from 25 to 2000 μm in height. The electric field or magnetic field system can be an electrostatic system disclosed in TW293787B or other systems that can generate the required energy.

The wavelength of the ultraviolet light used for irradiation curing is in the range of 10 to 400 nm. After irradiation, the ultraviolet-curable adhesive in the shape of microneedles is cured and cross-linked to form microneedles 400.

The drug is sprayed or dip-coated on the surface of the microneedles 400 to form drug-loaded microneedles, and the drug can be selected according to actual needs. During use, the microneedles penetrate the vessel wall, directly release the drug into the vessel wall, and then are withdrawn from the human body together with the drug-loaded microneedle balloon dilatation catheter.

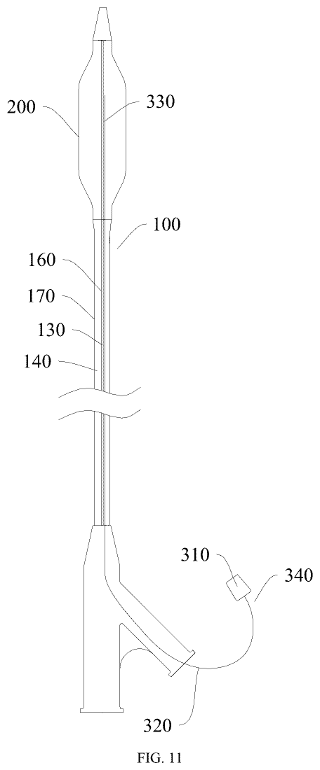

In one embodiment, 2 μL of acrylic resin adhesive (i.e., ultraviolet-curable adhesive) with a viscosity of 60,000 ps is applied at the interweaving points of the braided mesh (see FIG. 10), an electric field is applied to the outside of the balloon to form microneedle shapes, and then 275 nm UV light is irradiated for 15 s to cure and form the microneedles.

The drug-loaded microneedle balloon dilatation catheter provided in the present disclosure can effectively dilate calcified blood vessels, especially severely calcified blood vessels. When the balloon body is pressurized, the microneedles can be effectively pressed into the calcified blood vessel walls or the walls of the narrow digestive tract, cervix, airway, etc., and the drug can be slowly released for treatment.

The present disclosure provides two methods for preparing drug-loaded microneedle balloon dilatation catheters, wherein microneedles made using a substrate are bonded to the outer surface of the balloon body using a water-soluble adhesive, and the microneedles can be detached from the balloon body in the human body and remain embedded in the target tissue, making them particularly suitable for applications in the digestive tract and cervix among other parts. Microneedles prepared using ultraviolet-curable adhesive cannot be detached from the surface of the balloon body, but the drug coated on the surfaces of the microneedles still allows for effective drug delivery upon penetration into target tissues such as blood vessels.

The following provides a detailed description of a drug-loaded balloon catheter loaded with drug in the form of a solid coating referring to FIGS. 11 to 13. The drug coating loaded in the solid coating in FIGS. 11 to 13 contains a photosensitive material, i.e., a photosensitizer. From the perspective of improving the performance of the photosensitive material, modifications are made to the drug-loaded balloon catheter, mainly involving the preparation and loading methods of the photosensitive material.

A method for preparing a photosensitive material with blood vessel repair function includes:
step 1, protecting part of the amino groups of the polypeptide dendrimer; and
step 2, adding the polypeptide dendrimer with part of the amino groups protected and a naphthalimide compound to a mixed solution of an organic base and an organic solvent, reacting at 70 to 150° C. for 1 to 32 hours, followed by post-processing to obtain the photosensitive material;
wherein the organic base is at least one of N, N-diisopropylethylamine, sodium tert-butoxide, and potassium tert-butoxide; and
the organic solvent is at least one of isopropanol, hexafluoroisopropanol, methanol, tetrahydrofuran, dioxane, acetonitrile, ethyl acetate, dichloromethane, dimethyl sulfoxide, N, N-dimethylacetamide, N-methylpyrrolidone and hexamethylphosphoramide.

The naphthalimide compound is a photosensitive material. In the present disclosure, the naphthalimide compound undergoes a chemical reaction with the polypeptide dendrimer, connecting the naphthalimide compound to the polypeptide dendrimer through chemical bonds. Due to the regular structure of the polypeptide dendrimer, the naphthalimide compound is aggregated on the polypeptide dendrimer, improving the aggregation degree of the naphthalimide compound and the local light utilization efficiency.

After entering the human body, the polypeptide dendrimer modified with the naphthalimide compound can penetrate the extracellular matrix in the blood vessel wall, approaching the elastin and collagen in the blood vessel. Under light activation, an excited state is generated, inducing the cross-linking of proteins near the naphthalimide compound, thus playing a role in repairing the blood vessel.

Through the delivery and aggregation of the naphthalimide compound by the polypeptide dendrimer, the permeability of the naphthalimide compound in the blood vessel wall can be improved, so that it can enter the blood vessel wall more rapidly, achieving a more uniform distribution within a shorter time.

The polypeptide dendrimer is a dendrimer having amino groups and capable of undergoing nucleophilic substitution reactions, and is at least one of an arginine-based dendrimer, a lysine-based dendrimer, a glutamic acid-based dendrimer, and a proline-based dendrimer.

The lysine-based dendrimer is:

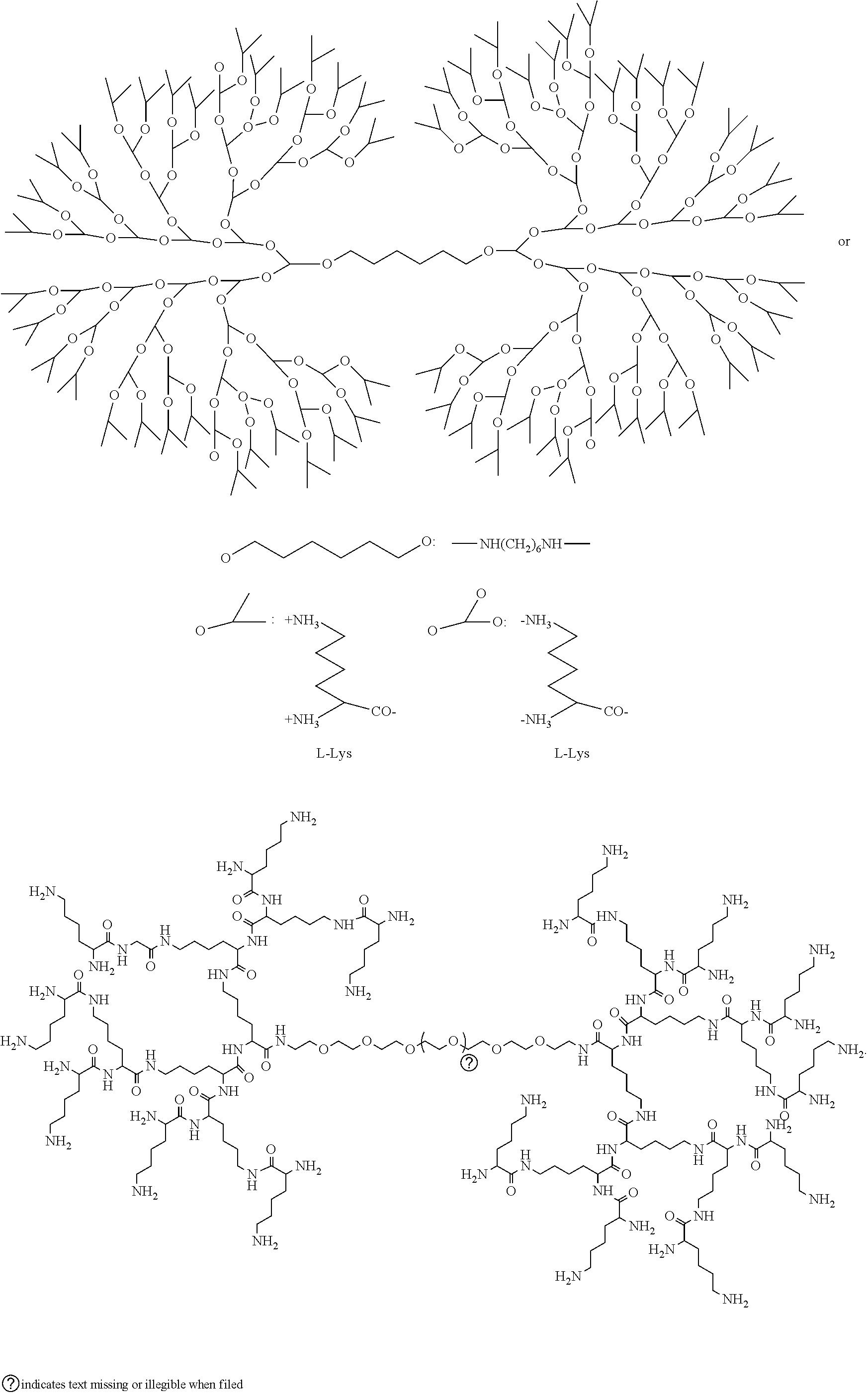

The lysine-based dendrimer is lysine-arginine dendrimer with the molecular structure shown below:

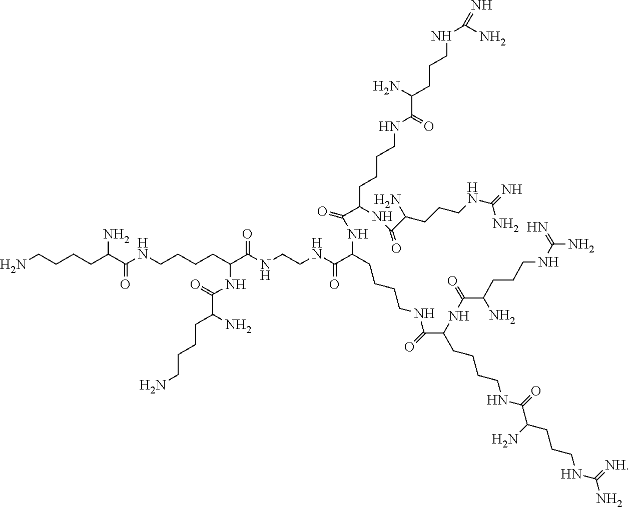 [SMILES: N=C(N)NCCCC(N)C(=O)NCCCCC(NC(=O)C(N)CCCNC(=N)N)C(=O)NCCCCC(NC(=O)C(CCCCNC(=O)C(N)CCCNC(=N)N)NC(=O)C(N)CCCNC(=N)N)C(=O)NCCNC(=O)C(CCCCNC(=O)C(N)CCCCN)NC(=O)C(N)CCCCN]

In step 1, part of the amino groups of the polypeptide dendrimer are protected. When the polypeptide dendrimer is a lysine-arginine dendrimer, the molecular structure after the amino groups are protected is as follows:

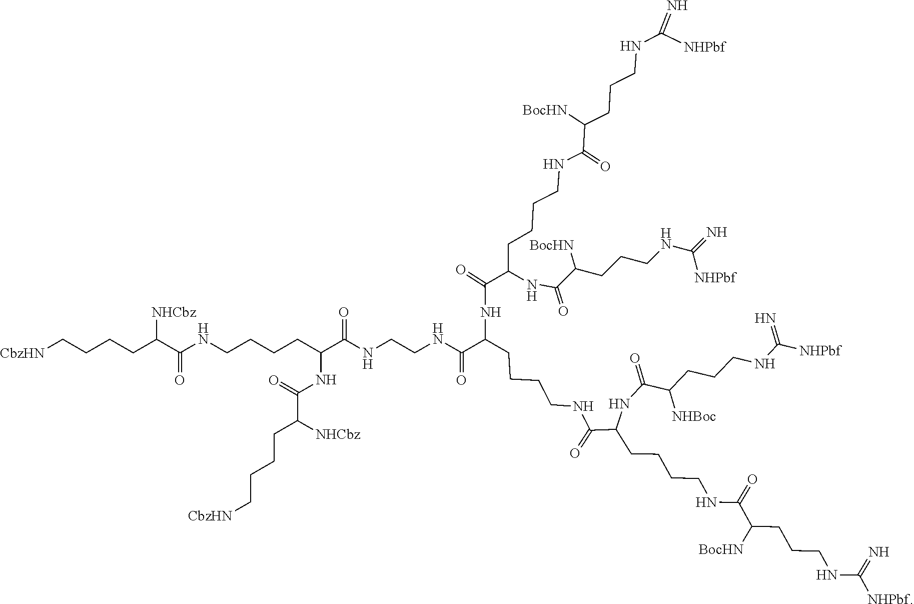

The naphthalimide compound is a derivative of 1,8-naphthalimide.

Derivatives of 1,8-naphthalimide include dimers, polymers, isomers and salt forms, for example, 1,8-naphthalimide dimer. When the polypeptide dendrimer is a lysine-arginine dendrimer, the structural formula of the photosensitive material is as follows:

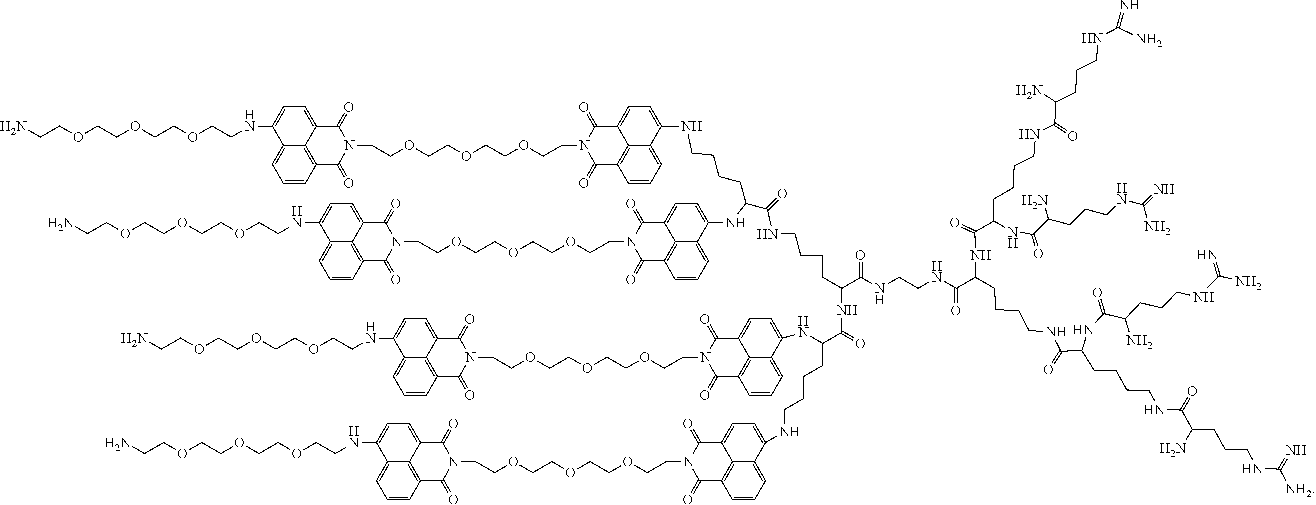 [SMILES: N=C(N)NCCCC(N)C(=O)NCCCCC(NC(=O)C(N)CCCNC(=N)N)C(=O)NCCCCC(NC(=O)C(CCCCNC(=O)C(N)CCCNC(=N)N)NC(=O)C(N)CCCNC(=N)N)C(=O)NCCNC(=O)C(CCCCNC(=O)C(CCCCNc1ccc2c3c(cccc13)C(=O)N(CCOCCOCCOCCN1C(=O)c3cccc4c(NCCOCCOCCOCCN)ccc(c34)C1=O)C2=O)Nc1ccc2c3c(cccc13)C(=O)N(CCOCCOCCOCCN1C(=O)c3cccc4c(NCCOCCOCCOCCN)ccc(c34)C1=O)C2=O)NC(=O)C(CCCCNc1ccc2c3c(cccc13)C(=O)N(CCOCCOCCOCCN1C(=O)c3cccc4c(NCCOCCOCCOCCN)ccc(c34)C1=O)C2=O)Nc1ccc2c3c(cccc13)C(=O)N(CCOCCOCCOCCN1C(=O)c3cccc4c(NCCOCCOCCOCCN)ccc(c34)C1=O)C2=O]

In step 1, protecting part of the amino groups of the polypeptide dendrimer includes the following steps:
step 1-1, protecting different amino groups of the polypeptide dendrimer with Cbz, Boc and Pbf; and
step 1-2, removing Cbz protection (removing Cbz protection in Pd/C methanol solution) to obtain a polypeptide dendrimer with part of its amino groups protected.

The structural formula of the Cbz protecting group is:

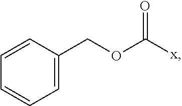

the structural formula of the Boc protecting group is

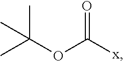

and the structural formula of the Pbf protecting group is

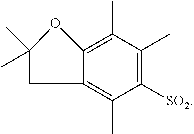

In step 2, the polypeptide dendrimer with part of its amino groups protected and the naphthalimide compound are added to a mixed solution of an organic base and an organic solvent, reacted at 70 to 100° C. for 4 to 32 hours, followed by post-processing to obtain the photosensitive material.

In step 2, the molar ratio of the polypeptide dendrimer to the naphthalimide compound is in the range of 1:3 to 6, preferably 1:4.

In step 2, the amount ratio of the organic base to the organic solvent is in the range of 40 mol: 30 to 90 mL.

In step 2, the amount ratio of the polypeptide dendrimer to the organic solvent is in the range of 4 mol: 30 to 90 mL.

In step 2, the preparation of the naphthalimide compound includes the following steps:
A. protecting one-side amino group of triethylene glycol amino group with Boc, to obtain one-side protected tert-butyl triethylene glycol amino group;
B. adding the one-side protected tert-butyl triethylene glycol amino group and 4-bromo-1,8-naphthalic anhydride into ethanol to form an ethanol solution, and heating the ethanol solution to 30 to 90° C. for 1 to 18 h to obtain the first product;
C. adding the one-side protected tert-butyl triethylene glycol amino group and 4-bromo-1,8-naphthalic anhydride into a mixed solution of N, N-diisopropylethylamine and dimethyl sulfoxide (DMSO), and heating the mixed solution to 70 to 150° C. for 1 to 32 hours to obtain the second product;
D. removing the Boc protection of the first product in trifluoroacetic acid and dichloromethane, adjusting the pH to be in the range of 6.5 to 8, stirring for 5 to 15 min, and extracting with dichloromethane to obtain the deprotected first product;
E. dissolving the deprotected first product and the second product in ethanol, heating to 30 to 90° C. and reacting for 1 to 18 h to obtain a naphthalimide compound.

The reaction equations from step B to step E are as follows.

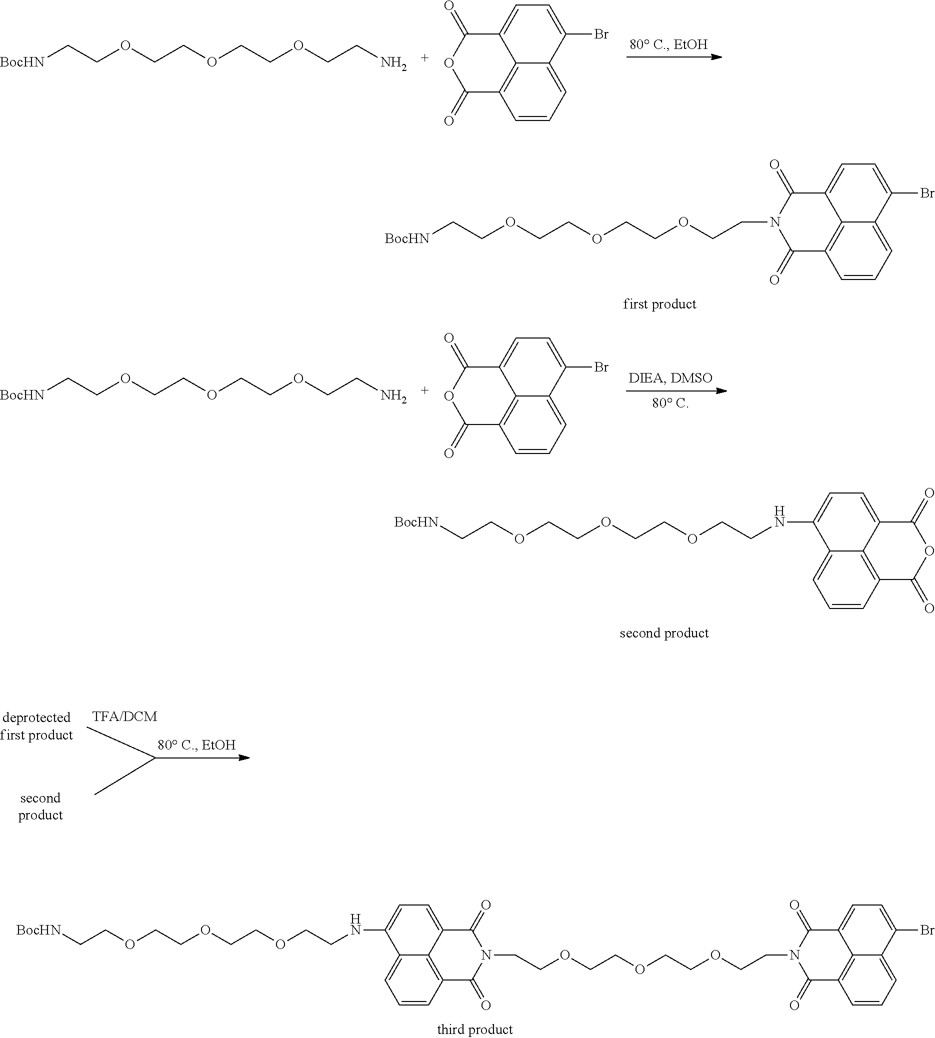

In step B, the molar ratio of the one-side protected tert-butyl triethylene glycol amino group to 4-bromo-1,8-naphthalic anhydride is in the range of 1:1 to 3, preferably in the range of 1:1 to 2.

In step B, the ratio of 4-bromo-1,8-naphthalic anhydride to ethanol is 1 mol: 0.8 to 1.1 L.

In step B, after the reaction, the product is washed with 30 mL of deionized water and filtered, and then vacuum dried to obtain the first product.

In step C, the molar ratio of the one-side protected tert-butyl triethylene glycol amino group and 4-bromo-1,8-naphthalic anhydride is 1:1.

In step C, the amount ratio of 4-bromo-1,8-naphthalic anhydride, N-diisopropylethylamine and dimethyl sulfoxide is: 1 mol: 2 mol: 7 to 8 L.

In step C, after the reaction, vacuum drying is performed to obtain a second product.

In step D, the pH is adjusted to 6.5 to 8 using a saturated sodium bicarbonate aqueous solution.

In step E, the molar ratio of the first product to the second product is 1:1.

In step 2, post-processing includes the following steps performed in sequence:
a. extracting with dichloromethane or chloroform;
b. separating and purifying the extracted organic phase by silica gel column chromatography or HPLC separation to obtain naphthalimide cross-linked polypeptide dendrimer;
c. removing the Boc and Pbf protections of the naphthalimide cross-linked polypeptide dendrimer; and
d. after rotary evaporation, adjusting the pH to be in the range of 6.5 to 8, stirring for 5 to 15 minutes, extracting with dichloromethane, and vacuum-drying to obtain the photosensitive material.

In step a, when extracting with dichloromethane or chloroform, the amount of dichloromethane or chloroform used is in the range of 15 to 50 mL.

In step c, the Boc and Pbf protections of the naphthalimide cross-linked polypeptide dendrimer are removed in a mixed solvent of trifluoroacetic acid and dichloromethane.

In step d, the pH is adjusted to be in the range of 6.5 to 8 using a saturated sodium bicarbonate aqueous solution.

Referring to FIG. 11, a balloon catheter system includes:
a tube body having opposing proximal and distal ends;
a balloon body, which is fixed to the distal end of the tube body; and
an optical fiber assembly inserted in the tube body and having a light-emitting portion extending to the position adjacent to the balloon body.

The surface of the balloon body 200 is loaded with an adjuvant and a photosensitizer in the form of a coating, and the photosensitizer is a polypeptide dendrimer modified with a naphthalimide compound.

The photosensitizer can activate collagen and elastin at a light wavelength in the range of 400 to 460 nm, inducing cross-linking.

The adjuvant includes an active drug and a sustained-release material encapsulating the active drug, and the active drug is at least one of paclitaxel, rapamycin, zotarolimus, tacrolimus, everolimus, temsirolimus, biolimus, docetaxel, protein-bound paclitaxel and protein-bound dexamethasone.

The surface of the balloon body 200 is loaded with an adjuvant and a photosensitizer in the form of coating, wherein the photosensitizer can activate the collagen and elastin of organs and/or tissues (such as blood vessels) at a light wavelength of 400 to 460 nm, inducing cross-linking, to form a micro-stent in situ. The adjuvant includes an active drug, which can be a drug for treating vascular diseases. The drug can be released into the blood vessels and/or on the blood vessel walls through the balloon body 200, and then absorbed by cells.

The dosage of the active drug is determined based on the lesion condition. However, during experiments, it was found that administering the predetermined dose did not achieve the expected therapeutic effect. Research revealed that this was due to the rapid release of the active drug from the surface of the balloon body (200) into the bloodstream. Additionally, the concentration trend of free active drug in the human body was not ideal, that is, the concentration of the free active drug decreased faster than the rate at which cells could absorb and utilize the active drug. Further, it was found that the reason for the too-rapid decrease in the concentration of the free active drug was that the free active drug was easily decomposed under the illumination with a wavelength in the range of 400 to 460 nm. When all the active drug was released into the blood, some of the free active drug could not be absorbed by cells in time and was decomposed by the light, resulting in inactivation. To solve this technical problem, the adjuvant further includes a sustained-release material encapsulating the active drug, which plays a role in protecting the active drug, reducing the loss rate of the active drug, and improving the utilization rate of the active drug.

The sustained-release material in the adjuvant is one of shellac, polyethylene glycol, magnesium stearate, povidone, alginic acid, ethyl cellulose, guar gum, gum arabic, hydroxypropyl methylcellulose, methylcellulose, polyvinylpyrrolidone, corn starch, calcium stearate, mineral oil, sodium stearyl fumarate, sodium benzoate, sodium lauryl sulfate, and stearic acid. More preferably, the sustained-release material is povidone K90.

As shown in FIG. 11, the tube body 100 can be a multi-lumen tube. For example, in one embodiment, the tube body 100 has at least a guidewire lumen 130, a perfusion lumen 140, and a receiving lumen. The guidewire lumen 130 is opened at both ends of the tube body 100 for the guidewire to pass through; one end of the perfusion lumen 140 is opened at the proximal end 120 of the tube body 100, and the other end is in communication with the interior of the balloon body 200. The balloon body 200 can be driven to expand by perfusing fluid in the perfusion lumen 140. After expansion, the coating on the surface of the balloon body 200 can be quickly released. The optical fiber assembly 340 is inserted into the receiving lumen, and its light-emitting portion 330 is adjacent to the balloon body 200. This adjacency mainly emphasizes that the light emitted by the optical fiber assembly 340 acts at a close distance to the balloon body 200, ensuring sufficient illumination coverage and intensity. This enables the photosensitizer to crosslink with the collagen fibers of the blood vessel wall, forming a vascular micro-stent with a certain level of support, allowing the blood vessel to remain expanded after the balloon body 200 is withdrawn.

The tube body 100 can include a plurality of tubes fitted over each other, and the interior of the tubes and/or the radial gaps between the inner and outer tubes 170 can be used to provide a guidewire lumen 130, a perfusion lumen 140 and a receiving lumen respectively. The perfusion lumen 140 and the receiving lumen can be a common or separate lumens.

For example, in one embodiment, the multiple tubes include an inner tube 160 and an outer tube 170, wherein the inner tube 160 provides a guidewire lumen 130, the gap between the inner tube 160 and the outer tube 170 provides a perfusion lumen 140, and the receiving lumen is provided by an independent tube or by the gap between the inner tube 160 and the outer tube 170, and wherein the independent tube is located in the gap between the inner tube 160 and the outer tube 170.

Alternatively, the receiving lumen can be provided by an extension tube located in the radial gap between the inner tube 160 and the outer tube 170, wherein the distal end 110 of the extension tube extends into the balloon body 200 and is fixed to the outer wall of the inner tube 160.

The material of the tube body 100 and the balloon body 200 can be nylon (PA), PEBAX, PEEK, PU, PVC, silicone, etc.

The optical fiber assembly 340 includes a light-emitting device 310 and an optical fiber body 320. The light-emitting device 310 is arranged outside the tube body 100. One end (proximal end 120) of the optical fiber body 320 is connected to the light-emitting device 310, and the other end (distal end 110) is inserted into the receiving lumen and extends to the position adjacent to the balloon body 200. The light-emitting portion 330 is provided at this end.

The optical fiber body 320 is a plastic optical fiber or a glass optical fiber, and the diameter of the optical fiber body 320 is in the range of 0.1 to 0.5 mm. The optical fiber body 320 can be movably placed in the tube body 100, or can be fixed in the tube body 100. Its fixation position can be adjusted based on the specific structure of the tube body 100, and the fixing method can be adhesive bonding or welding. For example, when the gap between the inner tube 160 and the outer tube 170 serves as the receiving lumen, the distal end 110 of the optical fiber body 320 can be fixed to the outer wall of the inner tube 160 or the inner wall of the outer tube 170 (see FIG. 11). If the receiving lumen is provided by the extension tube, the distal end 110 of the optical fiber body 320 extending out of the receiving lumen is fixed to the outer wall of the inner tube 160.

The photosensitizer and the active drug are released through the balloon body 200 catheter system, and the active drug is encapsulated with a sustained-release material with a light protection effect to reduce the loss rate of free active drug in the human body due to light decomposition, increase the utilization rate of active drug, and thus ensure the efficacy of active drug.

Before applying the coating on the surface of the balloon body, plasma treatment or surface coating with a hydrophilic material can be adopted.

After the surface of the balloon body is treated with plasma or coated with a hydrophilic material, the release of the drug on the balloon body will be delayed. The drug release rate of the hydrophilic-material-coated balloon body is slower than that of the plasma-treated one.

Loading the photosensitizer on the surface of the balloon body in the form of a coating includes the following steps: dispersing or dissolving the photosensitizer in a solvent to prepare a solution, and covering the surface of the balloon body with the solution.

The solvent is at least one of ethanol, acetic acid, acetone, butylated hydroxytoluene, methyl ethyl ketone, ethyl acetate, tetrahydrofuran and water.

The solvent is a mixture of ethanol, acetic acid and water, and the volume ratio of these components in the solvent is ethanol:acetic acid:water=80 to 90:19 to 9:1.

The solvent is a mixture of ethanol, acetic acid and water, and the volume ratio of these components in the solvent is ethanol:acetic acid:water=89:10:1.

In the solution, the concentration of the photosensitizer is in the range of 6.25 to 125 μM/mL, and more preferably, the concentration of the photosensitizer is in the range of 12.5 to 25 μM/mL.

The solution further includes an adjuvant, and the mass ratio of the adjuvant to the photosensitizer is in the range of 0.3 to 10, and more preferably, the mass ratio of the adjuvant to the photosensitizer is 1:1.

For the adjuvant, the mass ratio of active drug to sustained-release material is in the range of 1:1 to 20.

The mass ratio of photosensitizer to active drug is in the range of 1:0.2 to 5.

The solution further includes a stabilizer, which is at least one of an antioxidant and a Lewis acid.

The antioxidant is at least one of tromethamine and butylated hydroxytoluene. The mass ratio of the antioxidant to the photosensitizer is in the range of 0.05 to 1:100.

The cation of the Lewis acid is at least one of Na⁺, K⁺, Mg²⁺, and Ca²⁺. The molar ratio of the Lewis acid to the photosensitizer is in the range of 0.8 to 3.

The coating method includes spray coating and/or dipping coating, and the photosensitizer coating amount on the balloon surface is in the range of 0.0012 to 37.5 μM/mm². More preferably, the photosensitizer coating amount on the balloon surface is 0.05 μM/mm².

### Example 1 Preparation of Naphthalimide Compound

The preparation of the naphthalimide compound includes the following steps:
(1) protecting one-side amino group of the triethylene glycol amino group with Boc to obtain the one-side protected tert-butyl triethylene glycol amino group;
(2) adding 0.02 mol of the one-side protected tert-butyl triethylene glycol amino group and 0.02 mol of 4-bromo-1,8-naphthalic anhydride to 20 mL of ethanol to form an ethanol solution, and heating the ethanol solution to 80° C. and reacting for 22 h to obtain the first product;
(3) adding 0.02 mol of the one-side protected tert-butyl triethylene glycol amino group and 0.02 mol of 4-bromo-1,8-naphthalic anhydride to 0.04 mol of N, N-diisopropylethylamine and 150 mL of dimethyl sulfoxide to form a mixture, and heating the mixture to 80° C. and reacting for 1.5 h to obtain the second product;
(4) removing the Boc protection of the first product in trifluoroacetic acid and dichloromethane, adjusting the pH to be in the range of 6.5 to 8, stirring for 5 to 15 min, and extracting with dichloromethane to obtain the deprotected first product; and
(5) dissolving the deprotected first product and the second product in 55 mL of ethanol, heating to 80° C. and reacting for 2 h to obtain the naphthalimide compound (the third product).

### Example 2 Preparation of Photosensitive Material

A method for preparing a photosensitive material with blood vessel repair function includes the following steps:
(1) preparing the lysine-arginine dendrimer with a molecular structure as follows by the divergent method:

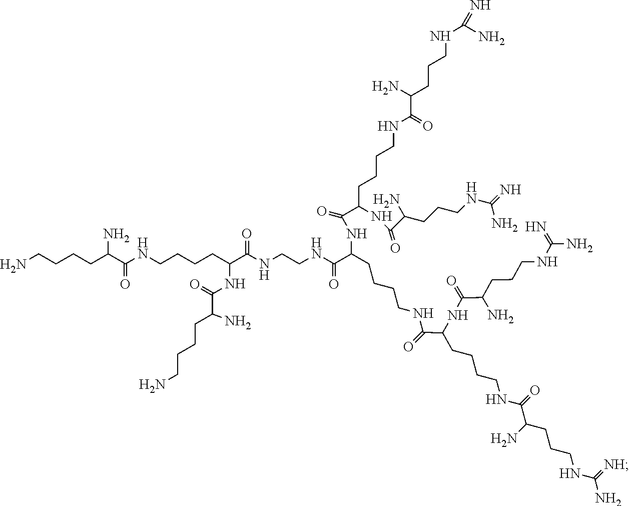 [SMILES: N=C(N)NCCCC(N)C(=O)NCCCCC(NC(=O)C(N)CCCNC(=N)N)C(=O)NCCCCC(NC(=O)C(CCCCNC(=O)C(N)CCCNC(=N)N)NC(=O)C(N)CCCNC(=N)N)C(=O)NCCNC(=O)C(CCCCNC(=O)C(N)CCCCN)NC(=O)C(N)CCCCN]

(2) protecting different amino groups of the lysine-arginine dendrimer with Cbz, Boc and Pbf, with a molecular structure as follows after the amino groups are protected:

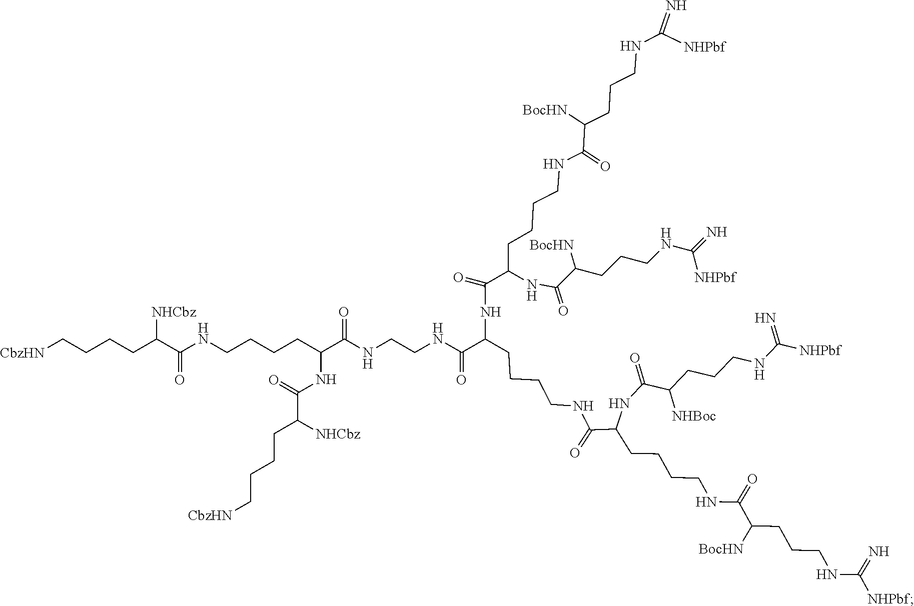

(3) removing Cbz protection in a Pd/C methanol solution to obtain a dendrimer with the Cbz protection removed;
(4) dissolving 1 mol of the dendrimer with the Cbz protection removed and 4 mol of the naphthalimide compound prepared in Example 1 in 90 mL of dimethyl sulfoxide (organic solvent), adding 40 mL of N, N-diisopropylethylamine (organic base), heating to 80° C., and stirring to react for 2 h;
(5) after the reaction, extracting with 30 mL of dichloromethane, separating the organic phase by column chromatography (silica gel: 200 to 300 mesh, eluent: ethyl acetate/ethanol=100:1), and vacuum-drying to obtain the photosensitive material protected by Boc and Pbf;
(6) adding the Boc and Pbf protected photosensitive material to a mixed solvent of trifluoroacetic acid and dichloromethane for deprotection; and
(7) after rotary evaporation, adding saturated sodium bicarbonate dropwise to adjust the pH to 7, stirring the mixture for 15 min, extracting with dichloromethane and then vacuum drying to obtain the photosensitive material with blood vessel repair function.

### Example 3 Preparation of Photosensitive Material Coating on the Balloon Surface

The photosensitive material with blood vessel repair function prepared in Example 2 was dissolved in an ethanol solution to prepare a 3.75×10⁻³ μmol/mL solution, which was sprayed on the surface of the balloon body at 5 μg/mm² and dried for 5 hours to obtain a balloon catheter containing a photosensitive material coating.

### Performance Standards

### 1. Permeability Characterization

Experimental group: a blood vessel was cut into 2*2 cm slices, with the inner surface facing upward and kept moist with 0.9% (by mass) physiological saline. 1 mg/mL of the photosensitive material with blood vessel repair function prepared in Example 1 was dripped onto the inner surface of the blood vessel, and the residual liquid on the inner surface of the blood vessel was wiped dry after a certain period of time, and the penetration depth in the blood vessel was measured using a laser confocal microscope.

Control group: a blood vessel was cut into 2*2 cm slices, with the inner surface facing upward and kept moist with 0.9% (by mass) physiological saline. 1 mg/mL of the third product in Example 1 was dripped onto the inner surface of the blood vessel, and the residual liquid on the inner surface of the blood vessel was wiped dry after a certain period of time, and the penetration depth in the blood vessel was measured using a laser confocal microscope.

TABLE 1

| | The time when the drug is observed |
|---|---|
| Group | in the adventitia of blood vessels |
| Experimental group | 18 s |
| Control group | 30 s |

As shown in Table 1, the photosensitive material in the experimental group had better permeability in the blood vessel wall. It can enter the blood vessel wall faster and tend to be evenly distributed in the blood vessel in a shorter time.

### 2. Blood Vessel Repair Effect

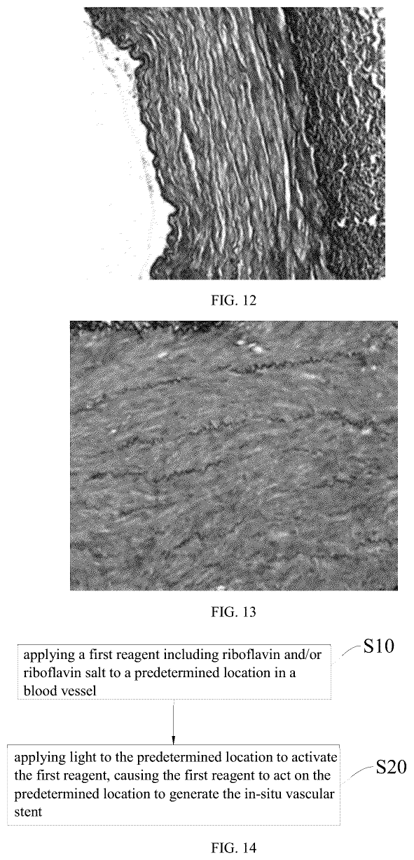

Experimental group: after taking a section of blood vessel and measuring its diameter, the balloon catheter with the intelligent photosensitive material coating prepared in Example 3 was pushed into the blood vessel, and the balloon body was inflated to the nominal pressure to dilate the blood vessel, and then the laser was turned on at 2.5 W for 1 minute before the balloon body was withdrawn. The dilated blood vessel section was taken for tissue staining to observe the cross-linking of elastic fibers and collagen fibers in the blood vessel. The cross-linking result is shown in FIG. 12, and the diameter change of the dilated blood vessel section was measured at the same time.

Control group: after taking a section of blood vessel and measuring its diameter, a balloon catheter with the 1,8-naphthalimide coating was pushed into the blood vessel, and the balloon body was inflated to the nominal pressure to dilate the blood vessel, and then the laser was turned on at 2.5 W for 1 minute before the balloon body was withdrawn. The dilated blood vessel section was taken for tissue staining to observe the cross-linking of elastic fibers and collagen fibers in the blood vessel. The cross-linking condition is shown in FIG. 12, and the diameter change of the dilated blood vessel section was measured at the same time.

TABLE 2

| Test result | Experimental group | Control group |
|---|---|---|
| The diameter of the blood | 2.6 | 2.72 |
| vessel before dilation (mm) | | |
| The diameter of the blood | 3.64 | 2.89 |
| vessel after dilation (mm) | | |

As shown in FIG. 12, tissue staining analysis showed that the collagen and elastin in the tunica media of the blood vessel in the experimental group were in a cross-linked state, indicating that the blood vessel maintained compliance. As shown in FIG. 13, most of the collagen and elastin in the tunica media of the blood vessel in the control group were broken, and the blood vessel lost compliance.

The balloon catheter system provided in the above embodiments improves the photosensitizer structure and coverage on the surface of the photodynamic balloon body, thereby increasing the utilization efficiency of the photosensitizer compound.

The following provides a detailed description of the drug-loaded balloon catheter utilizing solid coating and solution infiltration methods for drug loading, with reference to FIGS. 14 to 18. The drug loaded onto the drug-loaded balloon catheter described below primarily consists of riboflavin. The following will sequentially elaborate on the method for generating an in-situ vascular stent using the drug-loaded balloon catheter, the drug-loaded balloon catheter, and the photocurable balloon catheter system for generating the in-situ vascular stent.

Referring to FIG. 14, a method for generating an in-situ vascular stent according to one embodiment of the present disclosure includes:
step S10, applying a first reagent to a predetermined location in the blood vessel 70, wherein the first reagent includes riboflavin and/or a riboflavin salt;
step S20, applying light to the predetermined location to activate the first reagent, causing the first reagent to act on the predetermined location and form an in-situ vascular stent.

In step S10, the riboflavin salt may be riboflavin 5′-(dihydrogen phosphate) monosodium salt dihydrate, with the riboflavin (C₁₇H₂₀N₄O₆) content ranging from 74.0% to 79.0%.

When applying the first reagent, available methods include preparing a solution and directly delivering it to the predetermined location via an interventional device, or using approaches such as coating or solid embedding to deliver it to the predetermined location through the interventional device.

The interventional device may be, for example, an injection needle, a microporous balloon, or a tube 20 or the like. In addition, when applying the first reagent, blood flow may be blocked upstream and downstream of the predetermined location to achieve localized and precise drug delivery. In one embodiment, the active ingredient of the first reagent includes at least riboflavin, and when in solution, its concentration ranges from 0.2 to 60 mg/mL.

In another embodiment, the active ingredient of the first reagent is riboflavin, and its concentration ranges from 0.2 to 1.6 mg/mL, preferably from 0.2 to 1.2 mg/mL.

In another embodiment, the active ingredient of the first reagent is a riboflavin salt, and the total concentration ranges from 5 to 60 mg/mL when converted to riboflavin. In step S20, the wavelength of the applied light ranges from 300 to 700 nm. The intensity of the applied light ranges from 5 to 500 mW/cm², preferably from 100 to 500 mW/cm², and more preferably 500 mW/cm². The duration of applying light ranges from 0.1 to 30 minutes, preferably from 3 to 10 minutes, and more preferably 5 minutes.

The method for generating an in-situ vascular stent according to this embodiment can be used to deliver the drug to a target site (i.e., a predetermined location) of a tissue such as a blood vessel 70. The riboflavin and/or riboflavin salt in the first reagent is an anti-restenosis agent. After the first reagent is applied to the predetermined location, light is applied to activate the riboflavin, promoting its binding with collagen or other proteins on the vessel wall, thereby forming an in-situ vascular stent at the predetermined location. This in-situ vascular stent is an “endogenous” stent, which is more conducive to eliminating postoperative problems associated with implanted devices, such as poor compatibility, immunogenicity, thrombosis, and inflammation, compared to currently implanted metal stents or finished polymer stents.

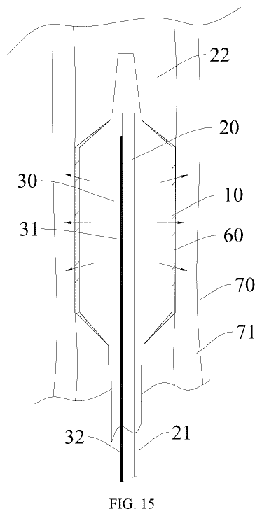

As shown in FIG. 15, an embodiment of the present disclosure further provides a drug-loaded balloon catheter based on photocuring, including a balloon body 10 and a tube body 20, wherein the balloon body 10 has an inflated state and a deflated state suitable for interventional delivery. The tube body 20 has a distal end 22 and an opposite proximal end 21, and the distal end 22 is connected to the balloon body 10 and is used to deliver fluid into the balloon body 10 to inflate the deflated balloon body 10.

The surface of the balloon body 10 is loaded with a drug coating 60. When the balloon body 10 is inflated, its surface acts on the blood vessel wall 71, and the drug can be applied to the blood vessel wall 71. In this embodiment, the active ingredients in the drug coating 60 include riboflavin and/or a riboflavin salt, which act as anti-restenosis agents. Under specific light excitation, these agents can bind to the collagen of the tissue or other proteins on the wall, thereby generating an in-situ vascular stent at the predetermined location. In order to generate an in-situ vascular stent, the drug-loaded balloon catheter further includes a light-guiding element 30, such as an optical fiber, etc. One end of the light-guiding element 30 is a light-emitting end 31 extending to the balloon body 10, and the other end is a light-incident end 32 extending through the tube body 20 to the proximal end 21. The light-emitting end 31 can emit light beams of a specific wavelength, which pass through the inner wall of the balloon body 10 and act on the blood vessel wall 71 (as shown by the arrow in FIG. 15), thereby activating riboflavin.

There are many methods for loading the drug coating 60. For example, in one embodiment, the method for loading the drug coating 60 includes:
preparing the drug coating 60 in advance, coating the solution onto the surface of the balloon body 10, and obtaining the drug coating 60 after drying.

The concentration of the active ingredient in the solution ranges from 0.2 to 1.2 mg/mL, calculated as riboflavin.

The coating amount per unit surface area of the balloon body affects the effectiveness of the in-situ vascular stent generation. In one embodiment, based on riboflavin, the coating amount per unit surface area of the balloon body is in the range of 0.05 to 20 μg/mm². This ensures the formation of a micro-stent with high bonding strength, effectively preventing restenosis of the blood vessel 70.

In one embodiment, the drug coating 60 further includes a carrier. The carrier may include at least one of polyethylene glycol, polyvinyl alcohol (PVA), polyethylene oxide (PEO), polysorbate, polyvinyl pyrrolidone, magnesium stearate, urea, tri-n-hexyl butyryl citrate, iopromide, ethyl cellulose, methylparaben, ethylparaben, tributyl acetyl citrate, glyceryl stearate, shellac, and pectin.

In one embodiment, the loading method of the drug coating 60 includes:
mixing the carrier, the active ingredient and the solvent to prepare a drug coating 60 solution.

For example, the carrier is dispersed in the solvent in advance, and then the active ingredient is added and mixed therewith.

In one embodiment, the loading method of the drug coating 60 includes:
preparing the active ingredient solution and the carrier solution separately in advance, coating the active ingredient solution on the surface of the balloon body and drying it, and after drying, then subsequently coating the carrier solution.

The carrier solution coating has the functions of protecting the drug coating 60 and releasing the active ingredients in a sustained manner.

The solvent of each above-mentioned solution includes at least one of water, methanol, ethanol, formic acid, acetic acid, acetonitrile, isopropanol, acetone, ethyl acetate, n-hexane, cyclohexane, dichloromethane, methyl acetate, butyl acetate, carbon tetrachloride, butanone and n-heptane.

The coating method can include spray or dipping coating. The coating methods of the active ingredient solution and the carrier solution may be different.

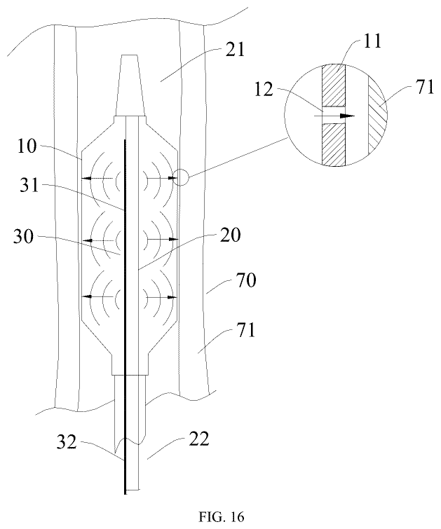

Referring to FIG. 16, a drug-loaded balloon catheter for an in-situ vascular stent according to one embodiment of the present disclosure includes a balloon body 10, a tube body 20 and a light-guiding element 30, wherein the balloon body 10 has an inflated state and a deflated state suitable for interventional delivery, and the balloon wall 11 of the balloon body 10 has a pore structure 12. The tube body 20 has a distal end 22 and an opposite proximal end 21, wherein the distal end 22 is connected to the balloon body 10. One end of the light-guiding element 30 is a light-emitting end 31 extending to the balloon body 10, and the other end is a light-incident end 32 extending through the tube body 20 to the proximal end 21.

The drug-loaded balloon catheter further includes a fluid, and the fluid contains riboflavin and/or a riboflavin salt. On the one hand, the fluid can be delivered to the balloon body 10 through the tube body 20 to inflate the deflated balloon body 10. On the other hand, the fluid in the balloon body 10 flows out through the pore structure 12, outputting to the surrounding environment of the balloon body 10 (as indicated by the fluid direction arrows in FIG. 16). The output fluid moves with the blood flow and enters the blood vessel wall 71. By controlling the light-emitting end 31 to emit light beams of a specific wavelength, these light beams pass through the balloon wall 11 of the balloon body 10 and act on the blood vessel wall 71 (shown by the wave lines in FIG. 16), thereby activating the riboflavin. This activation causes the riboflavin to bind to collagen or other proteins in the tissue, forming an in-situ vascular stent at the predetermined location.

The pore structure 12 mentioned above affects the rate at which the fluid is released into the surrounding environment of the balloon body 10. The pore size should not be too large to avoid the premature release (or loss) of the loaded drug before the balloon body 10 reaches the pressure required to dilate the blood vessel 70. Conversely, the pore size should not be too small, as it may result in a very slow drug release rate after the balloon body 10 inflates the blood vessel 70. In one embodiment, the pore size of the pore structure 12 ranges from 5 to 100 μm, preferably from 10 or 80 μm, and more preferably from 30 to 50 μm. The porosity of the surface of the balloon body 10 is between 30% and 80%, preferably between 40% and 70%, and more preferably between 45% and 60%.

The above-mentioned fluid is in the form of a solution, and its solvent is water, phosphate buffer solution, sodium chloride solution or physiological saline.

The concentration of the fluid ranges from 0.2 to 1.2 mg/mL based on total riboflavin, which can be released and fixed to the blood vessel wall 71 to form a stable in-situ vascular stent.

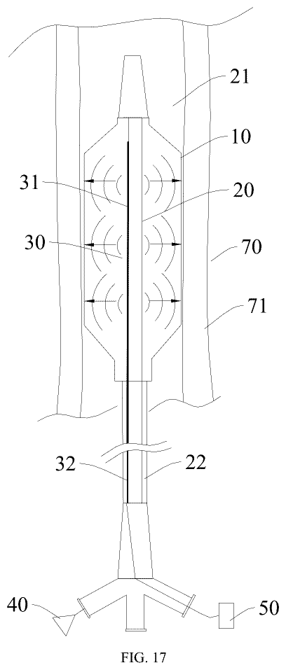

Referring to FIG. 17, a photocurable balloon catheter system for generating an in-situ vascular stent according to an embodiment of the present disclosure includes a balloon body 10, a tube body 20, a drug delivery device 40, a light-guiding element 30 and a light source device 50. The tube body 20 has a distal end 22 and an opposite proximal end 21. The balloon body 10 has a deflated state suitable for interventional delivery and an inflated state, and its balloon wall 11 has a pore structure 12 for fluid to pass through, and the balloon body 10 is connected to the distal end 22 of the tube body 20. The drug delivery device 40 is connected to the proximal end 21 of the tube body 20 and supplies fluid, and in this embodiment, the fluid contains riboflavin and/or riboflavin salt. One end of the light-guiding element 30 is a light-emitting end 31 extending to the balloon body 10, and the other end is a light-incident end 32 extending through the tube body 20 to the proximal end 21. The light source device 50 is connected to the light-incident end 32 of the light-guiding element 30 by an optical path.

During operation, the balloon body 10 in a deflated state is inserted into the target site (i.e., the predetermined location) of the blood vessel 70, and the fluid delivered by the drug delivery device 40 is delivered to the balloon body 10 through the tube body 20, so that the balloon body 10 is inflated to expose the pore structure 12, and the fluid is output from the pore structure 12 to the surrounding environment of the balloon body 10, and the output fluid moves with the blood flow and enters the blood vessel wall 71. The light source device 50 excites the light-emitting end 31 to emit light beams of a specific wavelength, thereby activating riboflavin. This activation causes the riboflavin to bind to collagen of the tissue or other proteins on the wall, forming an in-situ vascular stent at the predetermined location.

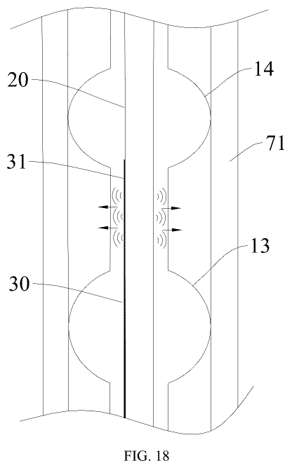

In one embodiment as shown in FIG. 18, the interventional device is a blocking balloon catheter, including at least two balloon bodies (a first balloon body 13 and a second balloon body 14), a tube body 20 and a light-guiding element 30. The first balloon body 13 and the second balloon body 14 are positioned on the upstream and downstream sides of the blood flow at the predetermined location, respectively. When in the inflated state, they can block blood flow on both sides to achieve localized precise drug delivery. The light-emitting end 31 of the light-guiding element extends into the tube body 20 and is located at a position corresponding to the predetermined location.

The present disclosure further provides the use of riboflavin and riboflavin salt in the preparation of in-situ vascular stent drug. After riboflavin and/or riboflavin salt are applied to the predetermined location, an in-situ vascular stent is formed at the predetermined location by light excitation.

A number of preparation examples are further provided below, and all reagents are commercially available.

### Preparation Example 1

Riboflavin was added to water and subjected to ultrasonic oscillation to prepare a riboflavin solution with a concentration of 0.5 mg/mL, which was used as the fluid.

### Preparation Example 2

32 mg of polyethylene glycol and 2 mL of acetic acid were fully mixed to prepare a solution, and 32 mg of riboflavin was added to the solution, followed by ultrasonic oscillation to prepare a drug coating solution. The drug coating solution was sprayed onto the surface of the balloon, with a spray amount of 3 μg/mm² based on riboflavin, to obtain a drug-loaded balloon.

### Preparation Example 3

32 mg of polyethylene glycol and 2 mL of acetic acid were fully mixed to prepare a solution, and 32 mg of riboflavin was added to the solution, followed by ultrasonic oscillation to prepare a drug coating solution. The drug coating solution was coated on the surface of the balloon by dip coating, with a coating amount of 4 μg/mm² based on riboflavin, to obtain a drug-loaded balloon.

### Preparation Example 4

32 mg of polyethylene glycol and 2 mL of acetic acid were fully mixed to prepare a carrier solution. 32 mg of riboflavin was added into water and subjected to ultrasonic oscillation to prepare a riboflavin solution. Firstly, the riboflavin solution was sprayed on the surface of the balloon and dried, and the spraying amount was 3 μg/mm² based on riboflavin; then, the carrier solution was sprayed and dried, and the spraying amount was 3 μg/mm² based on the carrier, to obtain a drug-loaded balloon.

### Preparation Example 5

32 mg of polyethylene glycol and 2 mL of acetic acid were fully mixed to prepare a carrier solution. 32 mg of riboflavin was added into water and subjected to ultrasonic oscillation to prepare a riboflavin solution. Firstly, the balloon was immersed in the riboflavin solution for 30 seconds, taken out and dried; then the balloon was immersed in the carrier solution for 30 seconds, taken out and dried to obtain a drug-loaded balloon.

### Preparation Example 6

32 mg of polyethylene glycol and 2 mL of acetic acid were fully mixed to prepare a carrier solution. 32 mg of riboflavin was added into water and subjected to ultrasonic oscillation to prepare a riboflavin solution. Firstly, the riboflavin solution was sprayed on the surface of the balloon and dried, with a spraying amount of 3 μg/mm² based on riboflavin; then, the balloon was immersed in the carrier solution for 30 seconds, taken out and dried to obtain a drug-loaded balloon.

### Preparation Example 7

32 mg of polyethylene glycol and 2 mL of ethanol were fully mixed to prepare a carrier solution. 32 mg of riboflavin was added into water and subjected to ultrasonic oscillation to prepare a riboflavin solution. Firstly, the riboflavin solution was sprayed on the surface of the balloon and dried, and the spraying amount was 3 μg/mm² based on riboflavin; then, the carrier solution was sprayed and dried, and the spraying amount was 3 μg/mm², to obtain a drug-loaded balloon.

### Preparation Example 8

32 mg of polyethylene glycol and 2 mL of ethanol were fully mixed to prepare a carrier solution. 32 mg of riboflavin was added into water and subjected to ultrasonic oscillation to prepare a riboflavin solution. Firstly, the balloon was immersed in the riboflavin solution for 30 seconds, taken out and dried; then the balloon was immersed in the carrier solution for 30 seconds, taken out and dried to obtain a drug-loaded balloon.

### Preparation Example 9

32 mg of polyethylene glycol and 2 mL of ethanol were fully mixed to prepare a carrier solution. 32 mg of riboflavin was added into water and subjected to ultrasonic oscillation to prepare a riboflavin solution. Firstly, the riboflavin solution was sprayed on the surface of the balloon and dried, and the spraying amount was 3 μg/mm² based on riboflavin; then, the balloon was immersed in the carrier solution for 30 seconds, taken out and dried to obtain a drug-loaded balloon.

### Application Example 1

A porcine peripheral blood vessel was immersed in PBS buffer. Balloons (with a pore size of 50 μm and a surface porosity of 50%) was introduced into the blood vessel. The fluid prepared in Preparation Example 1 was infused to expand the balloons from the compressed state to the inflated state at a pressure of 6 atm. While maintaining the pressure of the balloons, a light source device activated the light-guiding element to emit 450 nm light, with a photocuring duration of 5 minutes.

After irradiation, the balloons were withdrawn from the blood vessel. The residual amount of riboflavin on each balloon was then measured as follows: the balloons were immersed in a solvent that can dissolve riboflavin and subjected to ultrasonic treatment for 20 minutes. The concentration of riboflavin in the solvent was determined using high-performance liquid chromatography (HPLC), and the residual amount of riboflavin on the balloon surface was calculated. The residual rate (%) of riboflavin was obtained by comparing the residual amount to the initial coating amount. The results are shown in Table 1.

### Application Example 2

A porcine peripheral blood vessel was immersed in PBS buffer. Balloons prepared in Preparation Example 6 was pushed into the blood vessel. The compressed balloons were expanded by injecting fluid at a pressure of 6 atm. Then, while maintaining the pressure of the balloons, the light-guiding element emitted light with a wavelength of 450 nm, with a photocuring duration of 5 minutes.

After irradiation, the balloons were withdrawn from the blood vessel. The residual amount of riboflavin on each balloon was then measured as follows: the balloons were immersed in a solvent that can dissolve riboflavin and subjected to ultrasonic treatment for 20 minutes. The concentration of riboflavin in water was determined using high-performance liquid chromatography, and the residual amount of riboflavin on the balloon surface was calculated. The residual rate (%) of riboflavin was obtained by comparing the residual amount to the initial coating amount. The results are shown in Table 1.

A porcine peripheral blood vessel was immersed in PBS buffer. Balloons prepared in Preparation Example 9 was pushed into the blood vessel. The compressed balloons were expanded by injecting fluid at a pressure of 6 atm. Then, while maintaining the pressure of the balloons, the light-guiding element emitted light with a wavelength of 450 nm, with a photocuring duration of 5 minutes.

### Application Example 3

After irradiation, the balloons were withdrawn from the blood vessel. The residual amount of riboflavin on each balloon was then measured as follows: the balloons were immersed in a solvent that can dissolve riboflavin and subjected to ultrasonic treatment for 20 minutes. The concentration of riboflavin in water was determined using high-performance liquid chromatography, and the residual amount of riboflavin on the balloon surface was calculated. The residual rate (%) of riboflavin was obtained by comparing the residual amount to the initial coating amount. The results are shown in Table 1.

A porcine peripheral blood vessel was immersed in PBS buffer. Balloons without riboflavin were pushed into the blood vessel. The compressed balloons were expanded by injecting fluid at a pressure of 6 atm. Then, while maintaining the pressure of the balloons, the light-guiding element emitted light with a wavelength of 450 nm, with a photocuring duration of 5 minutes. After irradiation, the balloons were withdrawn from the blood vessel.

### Comparative Example 1

A porcine peripheral blood vessel was immersed in PBS buffer. Balloons prepared in Preparation Example 6 were pushed into the blood vessel. The compressed balloons were expanded by injecting fluid at a pressure of 6 atm. Then, while maintaining the pressure of the balloons, the light-guiding element was not activated for photocuring.

### Comparative Example 2

The balloons were then withdrawn from the blood vessel. The residual amount of riboflavin on each balloon was then measured as follows: the balloons were immersed in a solvent that can dissolve riboflavin and subjected to ultrasonic treatment for 20 minutes. The concentration of riboflavin in water was determined using high-performance liquid chromatography, and the residual amount of riboflavin on the balloon surface was calculated. The residual rate (%) of riboflavin was obtained by comparing the residual amount to the initial coating amount. The results are shown in Table 3.

TABLE 3
Residual Rate of Riboflavin on the Surface of Balloon

| Serial number | Riboflavin residual rate (%) |
|---|---|
| Application Example 1 | 9.5 |
| Application Example 2 | 15.4 |
| Application Example 3 | 17.8 |
| Comparative Example 2 | 35.2 |

Experimental research results using porcine peripheral blood vessels show that the lumen gain after angioplasty of the in-situ vascular stent with the photocurable balloon catheter and the riboflavin-treated balloon is significantly greater than that of angioplasty alone and that without photo-activation. These results also confirm that riboflavin can cross-link proteins under photo-activation, causing the treated arteries to exhibit a denser medial fiber network. The cross-linking of structural proteins such as collagen in the blood vessel wall helps preserve the natural vascular scaffold, which is beneficial for the treatment of damaged or diseased arteries.

In the present disclosure, riboflavin is transported or delivered to the blood vessel wall via an interventional device such as a balloon catheter. Then, riboflavin is activated by light, enabling it to cross-link with proteins and polypeptides in the blood vessel wall. As a result, an endogenous micro-stent is generated in situ on the blood vessel wall to replace the implantable stent. This can effectively reduce the formation of thrombus and immunogenicity. The micro-stent formed in situ can maintain the blood vessel in the dilated shape after angioplasty and prevent restenosis.

The technical features of the above embodiments can be arbitrarily combined, and not all possible combinations of the technical features of the above embodiments have been described for the sake of brevity of description. However, as long as there is no contradiction in the combination of these technical characteristics, such combination should be regarded as falling into the scope of this specification.

The above-described embodiments only illustrate several embodiments of the present disclosure, and the description thereof is specific and detail, but should not be construed as limiting the scope of the patent disclosure. It should be noted that, for those of ordinary skill in the art, several modifications and improvements can be made without departing from the concept of the present disclosure, all of which fall into the protection scope of the present disclosure.

1. A method for generating an in-situ vascular stent, comprising:
applying a first reagent comprising riboflavin and/or riboflavin salt to a predetermined location in a blood vessel; and
applying light to the predetermined location to activate the first reagent, causing the first reagent to act on the predetermined location to generate the in-situ vascular stent.

2. The method for generating an in-situ vascular stent of claim 1, wherein applying the first reagent comprises:
preparing a solution and delivering it to the predetermined location through an interventional device; or
using coating or solid embedding and delivering it to the predetermined location through the interventional device;
wherein when the first reagent is in the form of a solution, the first reagent has a concentration in a range of 0.2 to 60 mg/mL based on total riboflavin.

3. The method for generating an in-situ vascular stent of claim 1, wherein an active ingredient of the first reagent is riboflavin with a concentration in a range of 0.2 to 1.6 mg/mL.

4. The method for generating an in-situ vascular stent of claim 1, wherein a wavelength of the applied light is in a range of 300 to 700 nm.

5. The method for generating an in-situ vascular stent of claim 1, wherein an intensity of the applied light is in a range of 5 to 500 mW/cm².

6. The method for generating an in-situ vascular stent of claim 1, wherein a duration of applying light is in a range of 0.1 to 30 minutes.

7. The method for generating an in-situ vascular stent of claim 1, wherein an intensity of the applied light is in a range of 100 to 500 mW/cm² and a duration of applying light is in a range of 3 to 10 minutes.

8. A photocurable drug-loaded balloon catheter for implementing the method for generating an in-situ vascular stent of claim 1, comprising:
a tube body having opposite distal and proximal ends for delivering fluid into a balloon body;
the balloon body fixed to the distal end of the tube body and in communication with the tube body, the balloon body having a hollow structure and having an inflated state and a deflated state suitable for interventional delivery;
a drug coating loaded on an outer surface of the balloon body, an active ingredient in the drug coating comprising riboflavin and/or riboflavin salt; and
a light-guiding element having one end which is a light-emitting end extending to the balloon body and an other end which is a light-incident end extending to the proximal end through the tube body.

9. The drug-loaded balloon catheter of claim 8, wherein the drug coating is loaded by:
preparing a solution for drug coating in advance, coating the solution on the surface of the balloon body, and then drying it.

10. The drug-loaded balloon catheter of claim 8, wherein a coating amount per unit surface area of the balloon body is in a range of 0.05 to 20 μg/mm² calculated based on riboflavin.

11. A photocurable drug-loaded balloon catheter for implementing the method for generating an in-situ vascular stent of claim 1, comprising:
a balloon body having an inflated state and a deflated state suitable for interventional delivery, with a balloon body wall of a pore structure;
a tube body having opposite distal and proximal ends, the distal end being in communication with the balloon body;
a fluid for maintaining the balloon body in the inflated state, outputting to a surrounding environment of the balloon body through the pore structure, wherein the fluid contains riboflavin and/or riboflavin salt; and
a light-guiding element having one end as a light-emitting end extending to the balloon body and an other end as a light-incident end extending to the proximal end through the tube body.

12. The photocurable drug-loaded balloon catheter of claim 11, wherein a pore size of the pore structure is in a range of 5 to 100 μm.

13. The photocurable drug-loaded balloon catheter of claim 11, wherein a surface porosity of the balloon body is in a range of 30% to 80%.

14. The photocurable drug-loaded balloon catheter of claim 11, wherein the fluid is in a form of a solution, a solvent of which is water; and a concentration of the fluid is in a range of 0.2 to 60 mg/mL calculated based on total riboflavin.

15. Use of riboflavin and riboflavin salt in preparation of an in-situ vascular stent drug, comprising:
applying the riboflavin and/or riboflavin salt to a predetermined location and then generating an in-situ vascular stent at the predetermined location by photoexcitation.
